SEQUENCE LISTING (163 sequences)

SEQ ID NO: 1 - DNA, 22 bp, Artificial sequence
  siRNA sequence
aattgaagccaataaaccttcc

SEQ ID NO: 2 - DNA, 22 bp, Artificial sequence
  siRNA sequence
ggaaggtttattggcttcaatt

SEQ ID NO: 3 - DNA, 22 bp, Artificial sequence
  siRNA sequence
tgtgaaaataaacatttggatg

SEQ ID NO: 4 - DNA, 22 bp, Artificial sequence
  siRNA sequence
catccaaatgtttattttcaca

SEQ ID NO: 5 - DNA, 21 bp, Artificial sequence
  siRNA sequence
gtatttcaataaaaatgttga

SEQ ID NO: 6 - DNA, 21 bp, Artificial sequence
  siRNA sequence
tcaacatttttattgaaatac

SEQ ID NO: 7 - DNA, 22 bp, Artificial sequence
  siRNA sequence
cagttattaagaaataaactgc

SEQ ID NO: 8 - DNA, 22 bp, Artificial sequence
  siRNA sequence
gcagtttatttcttaataactg

SEQ ID NO: 9 - DNA, 22 bp, Artificial sequence
  siRNA sequence
aataaataaagcccagaagttt

SEQ ID NO: 10 - DNA, 22 bp, Artificial sequence
  siRNA sequence
aaacttctgggctttatttatt

SEQ ID NO: 11 - DNA, 21 bp, Artificial sequence
  siRNA sequence
gctcccctgagctgaaataaa

SEQ ID NO: 12 - DNA, 21 bp, Artificial sequence
  siRNA sequence
tttatttcagctcaggggagc

SEQ ID NO: 13 - DNA, 21 bp, Artificial sequence
  siRNA sequence
gtactgtgctgattcaataaa

SEQ ID NO: 14 - DNA, 21 bp, Artificial sequence
  siRNA sequence
tttattgaatcagcacagtac

SEQ ID NO: 15 - DNA, 19 bp, Artificial sequence
  siRNA sequence
aagaacacttttaataaac

SEQ ID NO: 16 - DNA, 19 bp, Artificial sequence
  siRNA sequence
gtttattaaaagtgttctt

SEQ ID NO: 17 - DNA, 21 bp, Artificial sequence
  siRNA sequence
cttcaaatagtggcaaataaa

SEQ ID NO: 18 - DNA, 21 bp, Artificial sequence
  siRNA sequence
tttatttgccactatttgaag

SEQ ID NO: 19 - DNA, 23 bp, Artificial sequence
  siRNA sequence
ttctactaataaagatggatcaa

SEQ ID NO: 20 - DNA, 23 bp, Artificial sequence
  siRNA sequence
ttgatccatctttattagtagaa

SEQ ID NO: 21 - DNA, 21 bp, Artificial sequence
  siRNA sequence
cttggactttaataaaaggga

SEQ ID NO: 22 - DNA, 21 bp, Artificial sequence
  siRNA sequence
tcccttttattaaagtccaag

SEQ ID NO: 23 - DNA, 21 bp, Artificial sequence
  siRNA sequence
cccttgtaacaataaaatcta

SEQ ID NO: 24 - DNA, 21 bp, Artificial sequence
  siRNA sequence
tagattttattgttacaaggg

SEQ ID NO: 25 - DNA, 21 bp, Artificial sequence
  siRNA sequence
acagcaataaagaggtgtcct

SEQ ID NO: 26 - DNA, 21 bp, Artificial sequence
  siRNA sequence
aggacacctctttattgctgt

SEQ ID NO: 27 - DNA, 22 bp, Artificial sequence
  siRNA sequence
ttcaataaacaagatcgtcagg

SEQ ID NO: 28 - DNA, 22 bp, Artificial sequence
  siRNA sequence
cctgacgatcttgtttattgaa

SEQ ID NO: 29 - DNA, 21 bp, Artificial sequence
  siRNA sequence
gtttcatagctaagaaataaa

SEQ ID NO: 30 - DNA, 21 bp, Artificial sequence
  siRNA sequence
tttatttcttagctatgaaac

SEQ ID NO: 31 - DNA, 21 bp, Artificial sequence
  siRNA sequence
catcatgggaataaattaatt

SEQ ID NO: 32 - DNA, 21 bp, Artificial sequence
  siRNA sequence
aattaatttattcccatgatg

SEQ ID NO: 33 - DNA, 20 bp, Artificial sequence
  siRNA sequence
gtttagtgtgtatcaataaa

SEQ ID NO: 34 - DNA, 20 bp, Artificial sequence
  siRNA sequence
tttattgatacacactaaac

SEQ ID NO: 35 - DNA, 22 bp, Artificial sequence
  siRNA sequence
gatcctaaaataaaccttggaa

SEQ ID NO: 36 - DNA, 22 bp, Artificial sequence
  siRNA sequence
ttccaaggtttattttaggatc

SEQ ID NO: 37 - DNA, 21 bp, Artificial sequence
  siRNA sequence
gctctggagagaaacaataaa

SEQ ID NO: 38 - DNA, 21 bp, Artificial sequence
  siRNA sequence
tttattgtttctctccagagc

SEQ ID NO: 39 - DNA, 21 bp, Artificial sequence
  siRNA sequence
taataaacccggacggaagcg

SEQ ID NO: 40 - DNA, 21 bp, Artificial sequence
  siRNA sequence
cgcttccgtccgggtttatta

SEQ ID NO: 41 - DNA, 21 bp, Artificial sequence
  siRNA sequence
ctgcaaagtctggtcaataaa

SEQ ID NO: 42 - DNA, 21 bp, Artificial sequence
  siRNA sequence
tttattgaccagactttgcag

SEQ ID NO: 43 - DNA, 22 bp, Artificial sequence
  siRNA sequence
caagtctaaatgatttaataaa

SEQ ID NO: 44 - DNA, 22 bp, Artificial sequence
  siRNA sequence
tttattaaatcatttagacttg

SEQ ID NO: 45 - DNA, 21 bp, Artificial sequence
  siRNA sequence
cttcaggtgtttaataaagat

SEQ ID NO: 46 - DNA, 21 bp, Artificial sequence
  siRNA sequence
atctttattaaacacctgaag

SEQ ID NO: 47 - DNA, 21 bp, Artificial sequence
  siRNA sequence
caagagaggaaataaaagcca

SEQ ID NO: 48 - DNA, 21 bp, Artificial sequence
  siRNA sequence
tggcttttatttcctctcttg

SEQ ID NO: 49 - DNA, 21 bp, Artificial sequence
  siRNA sequence
ctgggccgacagcagaataaa

SEQ ID NO: 50 - DNA, 21 bp, Artificial sequence
  siRNA sequence
tttattctgctgtcggcccag

SEQ ID NO: 51 - DNA, 21 bp, Artificial sequence
  siRNA sequence
aactatggttcccaataaaag

SEQ ID NO: 52 - DNA, 21 bp, Artificial sequence
  siRNA sequence
cttttattgggaaccatagtt

SEQ ID NO: 53 - DNA, 21 bp, Artificial sequence
  siRNA sequence
caaaatggaataaaaggcttg

SEQ ID NO: 54 - DNA, 21 bp, Artificial sequence
  siRNA sequence
caagccttttattccattttg

SEQ ID NO: 55 - DNA, 21 bp, Artificial sequence
  siRNA sequence
atagaaccaaataaacctacc

SEQ ID NO: 56 - DNA, 21 bp, Artificial sequence
  siRNA sequence
ggtaggtttatttggttctat

SEQ ID NO: 57 - DNA, 22 bp, Artificial sequence
  siRNA sequence
ctggtcccagcatgaaataaag

SEQ ID NO: 58 - DNA, 22 bp, Artificial sequence
  siRNA sequence
ctttatttcatgctgggaccag

SEQ ID NO: 59 - DNA, 21 bp, Artificial sequence
  siRNA sequence
ttaataaaactacctatctgg

SEQ ID NO: 60 - DNA, 21 bp, Artificial sequence
  siRNA sequence
ccagataggtagttttattaa

SEQ ID NO: 61 - DNA, 21 bp, Artificial sequence
  siRNA sequence
cccctcggggctggaataaaa

SEQ ID NO: 62 - DNA, 21 bp, Artificial sequence
  siRNA sequence
ttttattccagccccgagggg

SEQ ID NO: 63 - DNA, 21 bp, Artificial sequence
  siRNA sequence
caccatacacaagtaaataaa

SEQ ID NO: 64 - DNA, 21 bp, Artificial sequence
  siRNA sequence
tttatttacttgtgtatggtg

SEQ ID NO: 65 - DNA, 21 bp, Artificial sequence
  siRNA sequence
aataaaaactcctatttgcta

SEQ ID NO: 66 - DNA, 21 bp, Artificial sequence
  siRNA sequence
tagcaaataggagtttttatt

SEQ ID NO: 67 - DNA, 21 bp, Artificial sequence
  siRNA sequence
ttggataataaacctggctca

SEQ ID NO: 68 - DNA, 21 bp, Artificial sequence
  siRNA sequence
tgagccaggtttattatccaa

SEQ ID NO: 69 - DNA, 21 bp, Artificial sequence
  siRNA sequence
tatccaataaactaagtcggt

SEQ ID NO: 70 - DNA, 21 bp, Artificial sequence
  siRNA sequence
accgacttagtttattggata

SEQ ID NO: 71 - DNA, 21 bp, Artificial sequence
  siRNA sequence
agagggactccttccaataaa

SEQ ID NO: 72 - DNA, 20 bp, Artificial sequence
  siRNA sequence
ttattggaaggagtccctct

SEQ ID NO: 73 - DNA, 21 bp, Artificial sequence
  siRNA sequence
aataaaaggcattgacttaaa

SEQ ID NO: 74 - DNA, 21 bp, Artificial sequence
  siRNA sequence
tttaagtcaatgccttttatt

SEQ ID NO: 75 - DNA, 21 bp, Artificial sequence
  siRNA sequence
tgtcatatcaaataaagagag

SEQ ID NO: 76 - DNA, 22 bp, Artificial sequence
  siRNA sequence
ctctctttattttgatatgaca

SEQ ID NO: 77 - DNA, 22 bp, Artificial sequence
  siRNA sequence
ttcacagaataaaataaagcaa

SEQ ID NO: 78 - DNA, 23 bp, Artificial sequence
  siRNA sequence
ttgcttttattttattctgtgaa

SEQ ID NO: 79 - DNA, 21 bp, Artificial sequence
  siRNA sequence
atagttatagacctaaataaa

SEQ ID NO: 80 - DNA, 21 bp, Artificial sequence
  siRNA sequence
tttatttaggtctataactat

SEQ ID NO: 81 - DNA, 21 bp, Artificial sequence
  siRNA sequence
ggaacctgtatacacaataaa

SEQ ID NO: 82 - DNA, 21 bp, Artificial sequence
  siRNA sequence
tttattgtgtatacaggttcc

SEQ ID NO: 83 - DNA, 21 bp, Artificial sequence
  siRNA sequence
gctgcgcaaaattgcaataaa

SEQ ID NO: 84 - DNA, 21 bp, Artificial sequence
  siRNA sequence
tttattgcaattttgcgcagc

SEQ ID NO: 85 - DNA, 22 bp, Artificial sequence
  siRNA sequence
caaataaaatattttcaaagtc

SEQ ID NO: 86 - DNA, 22 bp, Artificial sequence
  siRNA sequence
gactttgaaaatattttatttg

SEQ ID NO: 87 - DNA, 21 bp, Artificial sequence
  siRNA sequence
agccatatgcaataaaataaa

SEQ ID NO: 88 - DNA, 21 bp, Artificial sequence
  siRNA sequence
tttattttattgcatatggct

SEQ ID NO: 89 - DNA, 23 bp, Artificial sequence
  siRNA sequence
aaggaataaacttgattatattg

SEQ ID NO: 90 - DNA, 23 bp, Artificial sequence
  siRNA sequence
caatataatcaagtttattcctt

SEQ ID NO: 91 - DNA, 21 bp, Artificial sequence
  siRNA sequence
atggaatcagcttttaataaa

SEQ ID NO: 92 - DNA, 21 bp, Artificial sequence
  siRNA sequence
tttattaaaagctgattccat

SEQ ID NO: 93 - DNA, 21 bp, Artificial sequence
  siRNA sequence
ccctcgatgaagcccaataaa

SEQ ID NO: 94 - DNA, 21 bp, Artificial sequence
  siRNA sequence
tttattgggcttcatcgaggg

SEQ ID NO: 95 - DNA, 21 bp, Artificial sequence
  siRNA sequence
gacaaattaaatctgaataaa

SEQ ID NO: 96 - DNA, 21 bp, Artificial sequence
  siRNA sequence
tttattcagatttaatttgtc

SEQ ID NO: 97 - DNA, 22 bp, Artificial sequence
  siRNA sequence
gtgaagccagaagccaaataaa

SEQ ID NO: 98 - DNA, 22 bp, Artificial sequence
  siRNA sequence
tttatttggcttctggcttcac

SEQ ID NO: 99 - DNA, 21 bp, Artificial sequence
  siRNA sequence
aataaaataactggcaaatat

SEQ ID NO: 100 - DNA, 21 bp, Artificial sequence
  siRNA sequence
atatttgccagttattttatt

SEQ ID NO: 101 - DNA, 24 bp, Artificial sequence
  siRNA sequence
gcatcctgagtttataataataaa

SEQ ID NO: 102 - DNA, 24 bp, Artificial sequence
  siRNA sequence
tttattattataaactcaggatgc

SEQ ID NO: 103 - DNA, 21 bp, Artificial sequence
  siRNA sequence
ttgtgttactgtgtcaataaa

SEQ ID NO: 104 - DNA, 21 bp, Artificial sequence
  siRNA sequence
tttattgacacagtaacacaa

SEQ ID NO: 105 - DNA, 21 bp, Artificial sequence
  siRNA sequence
atcaataaagagtaaattgtc

SEQ ID NO: 106 - DNA, 21 bp, Artificial sequence
  siRNA sequence
gacaatttactctttattgat

SEQ ID NO: 107 - DNA, 21 bp, Artificial sequence
  siRNA sequence
ttagtaataaaacattagtag

SEQ ID NO: 108 - DNA, 21 bp, Artificial sequence
  siRNA sequence
ctactaatgttttattactaa

SEQ ID NO: 109 - DNA, 21 bp, Artificial sequence
  siRNA sequence
tgaagtttaaataaagtttac

SEQ ID NO: 110 - DNA, 21 bp, Artificial sequence
  siRNA sequence
gtaaactttatttaaacttca

SEQ ID NO: 111 - DNA, 20 bp, Artificial sequence
  siRNA sequence
ttcaactgtaaataaagttt

SEQ ID NO: 112 - DNA, 20 bp, Artificial sequence
  siRNA sequence
aaactttatttacagttgaa

SEQ ID NO: 113 - DNA, 21 bp, Artificial sequence
  siRNA sequence
attgtttcagaacctaataaa

SEQ ID NO: 114 - DNA, 21 bp, Artificial sequence
  siRNA sequence
tttattaggttctgaaacaat

SEQ ID NO: 115 - DNA, 22 bp, Artificial sequence
  siRNA sequence
gaaattcaaaggtgaaaataaa

SEQ ID NO: 116 - DNA, 22 bp, Artificial sequence
  siRNA sequence
tttattttcacctttgaatttc

SEQ ID NO: 117 - DNA, 21 bp, Artificial sequence
  siRNA sequence
tttgccactgcaaacaataaa

SEQ ID NO: 118 - DNA, 21 bp, Artificial sequence
  siRNA sequence
tttattgtttgcagtggcaaa

SEQ ID NO: 119 - DNA, 22 bp, Artificial sequence
  siRNA sequence
caaataaatataagatctccag

SEQ ID NO: 120 - DNA, 22 bp, Artificial sequence
  siRNA sequence
ctggagatcttatatttatttg

SEQ ID NO: 121 - DNA, 21 bp, Artificial sequence
  siRNA sequence
aaccagtttccaataaaacgg

SEQ ID NO: 122 - DNA, 21 bp, Artificial sequence
  siRNA sequence
ccgttttattggaaactggtt

SEQ ID NO: 123 - DNA, 21 bp, Artificial sequence
  siRNA sequence
cccaaattccatgtcaataaa

SEQ ID NO: 124 - DNA, 21 bp, Artificial sequence
  siRNA sequence
tttattgacatggaatttggg

SEQ ID NO: 125 - DNA, 21 bp, Artificial sequence
  siRNA sequence
aaaggttttgaagtgaataaa

SEQ ID NO: 126 - DNA, 21 bp, Artificial sequence
  siRNA sequence
tttattcacttcaaaaccttt

SEQ ID NO: 127 - DNA, 22 bp, Artificial sequence
  siRNA sequence
ccatcagcatcaataaaacctc

SEQ ID NO: 128 - DNA, 22 bp, Artificial sequence
  siRNA sequence
gaggttttattgatgctgatgg

SEQ ID NO: 129 - DNA, 21 bp, Artificial sequence
  siRNA sequence
catctgaggaactggaataaa

SEQ ID NO: 130 - DNA, 21 bp, Artificial sequence
  siRNA sequence
tttattccagttcctcagatg

SEQ ID NO: 131 - DNA, 21 bp, Artificial sequence
  siRNA sequence
agaccaagaagcataaataaa

SEQ ID NO: 132 - DNA, 21 bp, Artificial sequence
  siRNA sequence
tttatttatgcttcttggtct

SEQ ID NO: 133 - DNA, 21 bp, Artificial sequence
  siRNA sequence
atgttacaataaagccttcct

SEQ ID NO: 134 - DNA, 21 bp, Artificial sequence
  siRNA sequence
aggaaggctttattgtaacat

SEQ ID NO: 135 - DNA, 21 bp, Artificial sequence
  siRNA sequence
tgtcaaataaatgagttcatc

SEQ ID NO: 136 - DNA, 21 bp, Artificial sequence
  siRNA sequence
gatgaactcatttatttgaca

SEQ ID NO: 137 - DNA, 22 bp, Artificial sequence
  siRNA sequence
aagattaacaacaggaaataaa

SEQ ID NO: 138 - DNA, 22 bp, Artificial sequence
  siRNA sequence
tttatttcctgttgttaatctt

SEQ ID NO: 139 - DNA, 21 bp, Artificial sequence
  siRNA sequence
aataaatatgaaacctcatat

SEQ ID NO: 140 - DNA, 21 bp, Artificial sequence
  siRNA sequence
atatgaggtttcatatttatt

SEQ ID NO: 141 - DNA, 20 bp, Artificial sequence
  siRNA sequence
ctggtatccttccaaataaa

SEQ ID NO: 142 - DNA, 20 bp, Artificial sequence
  siRNA sequence
tttatttggaaggataccag

SEQ ID NO: 143 - DNA, 23 bp, Artificial sequence
  siRNA sequence
cgcaatgattccatctaaataaa

SEQ ID NO: 144 - DNA, 23 bp, Artificial sequence
  siRNA sequence
tttatttagatggaatcattgcg

SEQ ID NO: 145 - DNA, 21 bp, Artificial sequence
  siRNA sequence
aacacagtcatgaataaagtt

SEQ ID NO: 146 - DNA, 21 bp, Artificial sequence
  siRNA sequence
aactttattcatgactgtgtt

SEQ ID NO: 147 - DNA, 21 bp, Artificial sequence
  siRNA sequence
ttcttgggtacgttcaataaa

SEQ ID NO: 148 - DNA, 21 bp, Artificial sequence
  siRNA sequence
tttattgaacgtacccaagaa

SEQ ID NO: 149 - DNA, 22 bp, Artificial sequence
  siRNA sequence
aataaaagaattgtctttctgt

SEQ ID NO: 150 - DNA, 22 bp, Artificial sequence
  siRNA sequence
acagaaagacaattcttttatt

SEQ ID NO: 151 - DNA, 21 bp, Artificial sequence
  siRNA sequence
aatcctgtcatataataaaaa

SEQ ID NO: 152 - DNA, 21 bp, Artificial sequence
  siRNA sequence
tttttattatatgacaggatt

SEQ ID NO: 153 - DNA, 21 bp, Artificial sequence
  siRNA sequence
gcatacagtctaaataataaa

SEQ ID NO: 154 - DNA, 21 bp, Artificial sequence
  siRNA sequence
tttattatttagactgtatgc

SEQ ID NO: 155 - DNA, 65 bp, Artificial sequence
  Oligonucleotide used for shRNA cloning
gatcccgcttacgctgagtacttcgattcaagagatcgaagtactcagcgtaagttttttggaaa

SEQ ID NO: 156 - DNA, 65 bp, Artificial sequence
  Oligonucleotide used for shRNA cloning
agcttttccaaaaaacttacgctgagtacttcgatctcttgaagcgaagtactcagcgtaagcgg

SEQ ID NO: 157 - DNA, 68 bp, Artificial sequence
  Oligonucleotide used for shRNA cloning
gatcccagctgcaataaacaagttaacttcaagagagttaacttgtttattgcagctttttttggaaa

SEQ ID NO: 158 - DNA, 68 bp, Artificial sequence
  Oligonucleotide used for shRNA cloning
agcttttccaaaaaaagctgcaataaacaagttaactctcttgaagttaacttgtttattgcagctgg

SEQ ID NO: 159 - DNA, 67 bp, Artificial sequence
  Oligonucleotide used for shRNA cloning
gatccgcctcaataaagcttgccttgttcaagagacaaggcaagctttattgaggcttttttggaaa

SEQ ID NO: 160 - DNA, 68 bp, Artificial sequence
  Oligonucleotide used for shRNA cloning
agcttttccaaaaaagcctcaataaagcttgcccttgtctcttgaacaaggcaagctttattgaggcg

SEQ ID NO: 161 - DNA, 21 bp, Artificial sequence
  anti-SV40 poly(A) signal shRNA
agctgcaataaacaagttaac

SEQ ID NO: 162 - DNA, 21 bp, Artificial sequence
  anti-HIV poly(A) signal shRNA
gcctcaataaagcttgccttg

SEQ ID NO: 163 - DNA, 21 bp, Artificial sequence
  anti-Luc ORF shRNA
cgcttacgctgagtacttcga

(19) United States
(12) Patent Application Publication
(10) Pub. No.: US 2008/0182813 A1
(43) Pub. Date: Jul. 31, 2008

(54) UNIVERSAL TARGET SEQUENCES FOR siRNA GENE SILENCING

(75) Inventors: Alik Honigman, Jerusalem (IL); Amos Panet, Mevaseret Zion (IL); Noam Levaot, Jerusalem (IL)

Correspondence Address: WINSTON & STRAWN LLP;PATENT DEPARTMENT, 1700 K STREET, N.W., WASHINGTON, DC, 20006, US

(73) Assignee: YISSUM RESEARCH DEVELOPMENT COMPANY OF THE HEBREW UNIVERSITY OF JERUSALEM

(21) Appl. No.: 12/058,373

(22) Filed: Mar. 28, 2008

Related U.S. Application Data
(62) Division of application No. 11/581,232, filed on Oct. 12, 2006.
Application No. 11/581,232 is a continuation of application No. PCT/IL2005/000437, filed on Apr. 21, 2005.
(60) Provisional application No. 60/564,214, filed on Apr. 22, 2004.

(51) Int. Cl. C07H 21/02; A61K 31/7088

(52) U.S. Cl. 514/44; 536/25.3

(57) ABSTRACT

The present invention provides a method for the production of a small interference RNA (siRNA) molecules for silencing the expression of a specific gene having AAUAAA as a polyadenylation signal site sequence. The method includes: a) identifying an oligonucleotide sequence of the specific gene, wherein the oligonucleotide sequence is about 15 to about 40 nucleotides in length and comprises (i) the polyadenylation signal site sequence and (ii) unique non-coding sequences flanking the polyadenylation signal site; and b) synthesizing oligonucleotide molecules having the oligonucleotide sequence (a), thereby obtaining an siRNA molecule for silencing the specific gene.

### CROSS-REFERENCE TO RELATED APPLICATIONS

The present application is a division of U.S. patent application Ser. No. 11/581,232, filed Oct. 12, 2006, now abandoned, which is a continuation of International Application No. PCT/IL2005/000437, filed Apr. 21, 2005, which, in turn, claims the benefit of Provisional Patent Application Ser. No. 60/564,214 filed Apr. 22, 2004, the entire content of each is expressly incorporated herein by reference thereto.

### FIELD OF THE INVENTION

The present invention relates to methods for reliably selecting and designing a sequence for efficient short interference RNA (siRNA) molecules. In particular, the present invention defines a target for siRNA silencing of cellular and viral genes.

### BACKGROUND OF THE INVENTION

There is a long-felt need in biotechnology and genetic engineering for targeted inhibition of gene expression. Although major efforts have been made to achieve this goal, a comprehensive solution to this problem is still needed in the art.

Classical genetic techniques have been used to isolate mutant organisms with reduced expression of selected genes. Although valuable, such techniques require laborious mutagenesis and screening programs, are limited to organisms in which genetic manipulation is well established (e.g., the existence of selectable markers, the ability to control genetic segregation and sexual reproduction), and are limited to applications in which a large number of cells or organisms can be sacrificed to isolate the desired mutation. Even under these circumstances, classical genetic techniques can fail to produce mutations in specific target genes of interest, particularly when complex genetic pathways are involved.

Many applications of molecular genetics require the ability to go beyond classical genetic screening techniques and efficiently produce a directed change in gene expression in a specified group of cells or organisms. Some such applications are knowledge-based projects in which it is of importance to understand what effects the loss of a specific gene product (or products) will have on the behavior of the cell or organism. Other applications are engineering based, for example cases in which it is important to produce a population of cells or organisms in which a specific gene product (or products) has been reduced or removed. A further class of applications is therapeutically based in which it would be valuable for a functioning organism (e.g., a human) to reduce or remove the amount of a specified gene product (or products). Another class of applications provides a disease model in which a physiological function in a living organism is genetically manipulated to reduce or remove a specific gene product (or products) without making a permanent change in the organism's genome.

In the last few years, advances in nucleic acid chemistry and gene transfer have inspired new approaches to engineer specific interference with gene expression.

RNA interference (RNAi) in Gene Silencing and Inhibition of Viral Replication

RNA Interference refers to the process of sequence-specific post-transcriptional gene silencing in higher eukaryotic cells mediated by short interfering RNAs (siRNAs) (Fire et al., Nature 391:806-811, 1998). The corresponding process in plants is commonly referred to as post-transcriptional gene silencing or RNA silencing. The process of post-transcriptional gene silencing is thought to be an evolutionarily conserved cellular defense mechanism used to prevent the expression of foreign genes and is commonly shared by diverse flora and phyla.

RNA interference, originally discovered in Caenorhabditis elegans by Fire and Mello (Fire et al., 1998), is a phenomenon in which double stranded RNA (dsRNA) reduces the expression of the gene to which the dsRNA corresponds. The phenomenon of RNAi was subsequently proven to exist in many organisms and to be a naturally occurring cellular process. The RNAi pathway can be used by the organism to inhibit viral infections, transposon jumping and to regulate the expression of endogenous genes. In these studies, the authors induced RNAi in non-mammalian systems using long double stranded RNAs.

However, most mammalian cells posses potent antiviral response mechanisms causing global changes in gene expression patterns in response to long dsRNA thus questioning the existence of RNAi in humans. As more information about the mechanistic aspects of RNAi was gathered, RNAi in mammalian cells was shown to exist as well.

In an in vitro system derived from Drosophila embryos, long dsRNAs were shown to be processed into shorter small interfering (si) RNA by a cellular ribonuclease containing RNaseIII motifs. Genetics studies carried out in C. elegans, N. crassa and A. thaliana have lead to the identification of additional components of the RNAi pathway. These genes include putative nucleases, RNA-dependent RNA polymerases and helicases. Several of these genes found in these functional screens are involved not only in RNAi but also in nonsense mediated mRNA decay, protection against transposon-transposition, viral infection, and embryonic development.

In general, it is thought that once the siRNAs are generated from longer dsRNAs in the cell by the RNaseIII like enzyme, the siRNA associates with a protein complex. The protein complex, also called RNA-induced silencing complex (RISC), then guides the smaller 21 base double stranded siRNA to the mRNA where the two strands of the double stranded RNA separate, the antisense strand associates with the mRNA and a nuclease cleaves the mRNA at the site where the antisense strand of the siRNA binds (Hammond et al., Nature Rev. Genet. 2:1110-1119, 2001). The mRNA is then subsequently degraded by cellular nucleases.

International PCT Publication No. WO 00/01846, describes certain methods for identifying specific genes responsible for conferring a particular phenotype in a cell using specific dsRNA molecules. International PCT Publication No. WO 01/29058 describes the identification of specific genes involved in dsRNA-mediated RNAi. International PCT Publication No. WO 99/07409, describes specific compositions consisting of particular dsRNA molecules combined with certain anti-viral agents. International PCT Publication No. WO 99/53050 describes certain methods for decreasing the phenotypic expression of a nucleic acid in plant cells using certain dsRNAs. International PCT Publication No. WO 01/49844 describes specific DNA constructs for use in facilitating gene silencing in targeted organisms.

International PCT Publications Nos. WO 02/055692, WO 02/055693, and EP 1144623 describe certain methods for inhibiting gene expression using RNAi. International PCT Publications Nos. WO 99/49029 and WO 01/70949, and AU 4037501 describe certain vectors expressing siRNA molecules. U.S. Pat. No. 6,506,559, describes certain methods for inhibiting gene expression in vitro using certain siRNA constructs that mediate RNAi.

Recent studies suggest that in mammalian cells exogenous siRNAs have been used to inhibit replication of different viruses, such as hepatitis B and C, polio virus and HIV 1 (Hamasaki, K., et al., FEBS Lett. 543:51-54).

U.S. Pat. No. 6,667,152 discloses methods for selective inactivation of viral replication by determining whether a potential agent interacts with a virus or cellular component which allows or prevents preferential translation of a virus RNA compared to a host RNA under virus infection conditions.

U.S. Pat. No. 5,990,388 discloses methods for displaying resistance to viruses and viroids in transgenic plants and animals expressing dsRNA-binding protein.

U.S. Pat. Nos. 5,063,209 and 4,820,696 disclose methods for modulation of AIDS-virus-related events by double-stranded RNAs.

U.S. Pat. No. 5,681,747 discloses methods for inhibiting human-PKCα expression with an oligonucleotide specifically hybridizable to a portion of the 3′-untranslated region of PKCα.

Konishi et al., (Hepatology, 38(4): 842-850, 2003) have shown that siRNA targeted against the polyadenylation (PA), precore (PreC) and surface (S) regions in the HBV genome can inhibit HBV replication. However the region of polyadenylation signal in HBV targeted by siRNA is different from the consensus sequence of the polyadenylation signal site (AAUAAA). Furthermore, there is no explanation in this publication as to why the polyadenylation signal site was chosen as a target and there is no general conclusion about using this region as a universal target.

Despite the rapid progress in this field, application of siRNA technology for whole-genome phenotypic screening faces a major obstacle that derives from the difficulty to predict the effectiveness of a selected RNA sequence as a target for siRNA mediated inhibition. Such molecules require assaying to determine whether they possess this activity, which can be time consuming. Thus, it would be advantageous to be able to generate database of small, double-stranded RNA molecules, which may mediate RNA interference.

Effective siRNA target sequences within a gene are limited and may depend on a combination of several variables. Likely variables include target mRNA stem and loop secondary structures, target RNA interaction with binding proteins, and sequence dependencies for the formation of functional “RNA induced silencing complex”.

Definition of an efficient target for siRNA is yet a major obstacle in the design of a siRNA construct. Although computer programs for the prediction of preferred target sites for siRNA were designed, the finding of an optimal target sequence is still a laborious, expensive and time-consuming process. Another obstacle in the development of siRNA for gene silencing is the emergence of resistant mutants. The degenerative nature of the genetic code, leading to silent mutations, and non-lethal changes of amino acids in a protein, leads to selection of resistance to siRNA. This phenomenon is amplified in fast replicating genomes such as viruses. Genetic signals in regulatory non-coding regions such as the poly(A) signal, may be less tolerant to mutations, and thus are less susceptible to escape mutations.

There is an unmet need for improved methods for designing and generating effective dsRNA molecules that may serve to silence or inhibit target genes, in a manner that is specific, safe and effective, and avoids the need to screen empirically a large number of candidate molecules.

### SUMMARY OF THE INVENTION

The present invention provides compositions and methods for inhibiting expression of a target gene in a cell. Inhibition is specific in that a nucleotide sequence from a portion of the target gene is chosen to produce inhibitory RNA. The process comprises introduction of double-stranded short interference RNA into the cells and reducing the expression of the corresponding messenger RNA in the cells. This process is advantageous compared to compositions or methods as are known in the art, in several respects: (1) effectiveness in producing inhibition of gene expression, (2) specificity to the targeted gene, and (3) general siRNA design applicability while enabling specific inhibition of many different types of target genes.

The present invention for the first time discloses the finding that a consensus sequence present in the polyadenylation (Poly(A)) signal site of expressed genes provides a universal sequence that is useful to design effective short interfering RNAs (siRNAs) without resorting to laborious and time-consuming efforts required to identify appropriate targets within the coding sequences of the gene. The polyadenylation signal site of eukaryotic mRNAs commonly comprises a consensus sequence of 6 nucleotides that are located 10-30 nucleotides upstream of the poly(A) tail. This consensus sequence enables the universal design of appropriate siRNAs, and when combined with unique sequences present adjacent to the consensus sequence, constitute a molecule that has a consensus universal part (enabling easy design) and a unique part (enabling specific gene silencing).

According to a first aspect the present invention provides a small interference RNA (siRNA) molecule comprising a first segment comprising a consensus sequence of the polyadenylation signal (poly(A)) site or a fragment thereof, and a second segment comprising unique non-coding sequences flanking said consensus sequence.

The term “flanking” refers to sequences that are upstream adjacent, downstream adjacent, or both upstream and downstream of the consensus sequence.

According to one embodiment, the siRNA comprises 6 nucleotides of the Poly(A) signal site consensus sequence AAUAAA. However, it should be appreciated that the present invention also encompasses a Poly(A) signal site that may comprise shorter or longer number of nucleotides.

According to another embodiment, the siRNA of the present invention further comprises 9 to 34 unique flanking nucleotides. The unique flanking sequences provide specificity of the siRNA to the target gene.

According to one embodiment, the siRNA comprises a total of about 15 to about 40 nucleotides, preferably the siRNA comprises from about 18 to about 25 nucleotides corresponding to at least a part of the consensus sequence of the Poly(A) signal site of the target gene. It is to be understood that said siRNA can be designed by bio-informatic programs to predict the optimal length of the flanking sequences to be used on either end of the consensus sequence of the polyadenylation signal site.

According to certain embodiments, the siRNA is capable of inhibiting the expression of a target gene in a cell. The target gene is selected from the group consisting of an endogenous cellular gene, an exogenous gene which is not present in the normal cellular genome and a gene of an infectious agent such as a viral gene.

According to other embodiments, the target gene of the present invention is of mammalian origin, avian origin, insect origin, plant origin, yeast origin, fungi origin, parasite origin, or viral origin. According to other embodiments the siRNA is of human origin. According to some embodiments the target gene is expressed in a tumor cell.

According to certain preferred embodiments the siRNA is capable of inhibiting the expression of a target gene by at least 50%, preferably by at least 65%, more preferably by at least 75% and most preferably by at least 95%. According to some embodiments 99% or more inhibits the expression of the target gene.

According to certain preferred embodiments the siRNA is useful for abrogation of virus propagation and for abrogation of cell proliferation.

According to certain embodiments the sequence of the siRNA is identical to the corresponding target gene sequence. According to another embodiment the sequence of the siRNA of the present invention comprises at least one mismatch pair of nucleotides. Preferably, the siRNA sequence comprises no more than two mismatch pairs of nucleotides.

According to certain preferred embodiments the siRNA comprising a sequence selected from the group consisting of any one of SEQ ID Nos: 1 to 160.

According to another aspect the present invention provides an expression vector capable of expressing the above siRNAs. The expression vector comprises control elements (promoter/enhancers) operably linked to sequences coding for the siRNA. Typically, these sequences are capable of coding of both the sense and the anti sense strands of the siRNA.

According to a further aspect the present invention comprises a siRNA expression vector wherein the siRNA comprises a first segment comprising a consensus sequence of the polyadenylation signal site or a fragment thereof, and a second segment comprising unique non-coding sequences flanking said consensus sequence.

According to yet another aspect the present invention provides a pharmaceutical composition comprising as an active ingredient a siRNA molecule comprising a first segment comprising a consensus sequence of the polyadenylation signal site or a fragment thereof, and a second segment comprising unique non-coding sequences flanking said consensus sequence and a pharmaceutically acceptable carrier.

According to still another aspect the present invention provides a pharmaceutical composition comprising as an active ingredient a siRNA expression vector, wherein the siRNA comprises a first segment comprising a consensus sequence of the polyadenylation signal site or a fragment thereof, and a second segment comprising unique non-coding sequences flanking said consensus sequence.

According to another aspect the present invention comprises generating a siRNA library comprising of a plurality of siRNA molecules comprising a first segment comprising a consensus sequence of the polyadenylation signal site or a fragment thereof, and a second segment comprising unique non-coding sequences flanking said consensus sequence.

Preferably, the siRNA library is directed against targets selected from the group consisting of mRNA splice variants, functionally related mRNAs or the total mRNAs present in a cell.

According to one embodiment, generating said siRNA library for a selected group of genes, comprises the following steps:
a) identifying oligonucleotide sequences corresponding to the sequences flanking the poly(A) signal site of selected genes;
b) preparing oligonucleotides comprising about 20 to about 25 nucleotides corresponding to the sequences flanking the poly(A) signal site for the selected genes;
c) utilizing said oligonucleotides of about 20 to about 25 nucleotides as primers for PCR of cDNA libraries or of a genomic DNA library; and
d) cloning the resulting PCR products into siRNA expression vectors.

According to some embodiments, identifying the oligonucleotide sequences utilizes data from a gene bank.

According to one embodiment, generating a random siRNA library corresponding to total mRNA in a given cell comprises the following steps:
a) isolating total mRNA from a biological sample;
b) preparing at least 32 oligonucleotide primers comprising at least 16 oligo-dT primers that differ from each other in at least one nucleotide located in the 3′ end of each primer and at least 16 additional oligonucleotide primers consisting of the poly(A) signal that differ from each other in at least one nucleotide located at the 3′ end of each oligonucleotide;
c) utilizing said at least 32 oligonucleotides as primers for PCR of mRNA extracts obtained in (a); and
d) cloning the resulting PCR products into siRNA expression vectors.

According to alternative embodiments the siRNAs are chemically synthesized to generate a siRNA library.

According to another aspect the present invention concerns a method for the production of siRNAs for silencing the expression of a specific gene, the method comprising the steps of:
a) identifying one or more oligonucleotide sequences corresponding to about 15 to about 40 nucleotides comprising the sequences of the Poly(A) signal site of the specific gene; and
b) synthesizing the oligonucleotides of (a) thereby obtaining siRNAs for silencing said gene.

According to some embodiments, identifying the oligonucleotide sequences utilizes data from a gene bank.

It should be appreciated that the orientation of the flanking unique sequence in respect to the consensus sequence (5′, 3′ or both 5′ and 3′) may vary and the total size of the siRNA may also vary between 15-40 oligonucleotides. Therefore the above method can result in a plurality of candidate siRNAs. It should be appreciated that some of the siRNAs can have better gene silencing properties than others. In order to select the best candidates from the plurality of candidate siRNAs, the siRNAs can be introduced into the cell and the level of expression of the gene determined (by mRNA determination, protein level determination or functional determination). Those siRNA which caused the highest percentage of silencing are the optimal siRNAs for silencing the gene.

According to another aspect the present invention provides a method for inhibiting the expression of a target gene in a cell of an organism comprising the step of introducing into the cell an effective amount of a siRNA to attenuate the expression of the target gene wherein the siRNA comprises a first segment comprising a consensus sequence of the polyadenylation signal site or a fragment thereof, and a second segment comprising unique non-coding sequences flanking said consensus sequence. It should be appreciated that the method of the present invention is highly advantageous in therapy in which transcription and/or translation of a mutated or other detrimental gene should be attenuated.

Further aspects of the present invention provides a method for preventing or treating a disease or disorder, wherein a beneficial therapeutic effect is evident due to the silencing of at least one gene, said method comprising the step of administering to a subject in need thereof a pharmaceutical composition comprising a therapeutically effective amount of a siRNA for the at least one gene, wherein the siRNA molecule comprises at least a part of the consensus sequence of the polyadenylation signal site and at least a second part of unique non-coding sequences flanking said consensus sequence of the polyadenylation signal.

According to some preferred embodiments the present invention further provides methods for preventing or treating a disease or disorder, comprising administering to a subject in need thereof a pharmaceutical composition comprising a therapeutically effective amount of a siRNA expression vector, as disclosed herein above.

According to one embodiment the transfection of siRNA molecules attenuates expression of a selected target gene within a cell ex-vivo. In certain embodiments the transfection or infection of siRNA expression vector attenuates expression of a selected target gene within a cell ex-vivo.

According to some embodiments the delivery of siRNA molecules attenuates expression of a selected target gene within an organism in-vivo. In certain embodiments the delivery of siRNA expression vector attenuates expression of a selected target gene within an organism in-vivo.

According to some embodiments the methods of the present invention is useful to treat a disease or disorder selected from a group consisting of a neoplastic disease, a hyperproliferative disease, angiogenesis, chronic inflammatory diseases and chronic degenerative diseases.

The compositions and methods of the present invention are useful in treating any type of cancer including solid tumors and non-solid tumors. The solid tumors are exemplified by CNS tumors, liver cancer, colorectal carcinoma, breast cancer, gastric cancer, pancreatic cancer, bladder carcinoma, cervical carcinoma, head and neck tumors, vulvar cancer and dermatological neoplasms including melanoma, squamous cell carcinoma and basal cell carcinomas. Non-solid tumors include lymphoproliferative disorders including leukemias and lymphomas.

According to some embodiments the methods are useful to treat a neoplastic disease in a human subject.

In certain embodiments the siRNA or the siRNA expression vector is injected directly to the tumor site. Alternatively, the siRNA is administered systemically.

According to another aspect the present invention provides a method of examining the function of a gene in a cell or organism comprising the steps of:
a) introducing into a cell or to an organism a double-stranded RNA that corresponds to at least one mRNA of the gene comprising a first consensus sequence corresponding to at least a part of the polyadenylation signal site and a second unique sequence corresponding to about 9-34 contiguous bases from the region adjacent to either end of the consensus sequence of the Poly(A) signal site;
b) maintaining the cell or organism produced in (a) under conditions which preserve viability; and
c) observing the phenotype of the cell or organism produced in (b) and, optionally, comparing the phenotype observed to that of a control cell or control organism which does not comprise said double-stranded RNA, thereby providing information about the function of the gene.

Yet another embodiment of the invention relates to a method for the production of a small interference RNA (siRNA) molecule for silencing the expression of a specific gene having AAUAAA as a polyadenylation signal site sequence, the method comprising the steps of:

a) identifying an oligonucleotide sequence of the specific gene, wherein said oligonucleotide sequence is about 15 to about 40 nucleotides in length and comprises
(i) said polyadenylation signal site sequence and
(ii) unique non-coding sequences flanking said polyadenylation signal site; and

b) synthesizing oligonucleotide molecules having said oligonucleotide sequence (a), thereby obtaining an siRNA molecule for silencing said specific gene.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Details of the embodiments of the present invention are illustrated in the appended drawings wherein:

FIG. 1 shows a schematic presentation of the poly(A) region conservation in the HIV-1 genome. The numbers on the X-axis represent the position on the HIV-1 genomic RNA. Number of copies (the Y axis) refers to the number of HIV-1 genomes that share a unique 21-bases-long sequence. The poly(A) region sequence in the R region is marked with arrows and its level of conservation relative to other sequences is presented by a horizontal dashed line. A diagram of the HIV genome is presented below according to each gene's relative position.

FIGS. 2A-2B are schematic presentations of the siRNA expressing vectors. FIG. 2A shows schematic presentation of the pSilencer 2.0 vector that was used to construct pSA-SV, and pSA-HIV vectors expressing shRNA targeting the SV-40 poly(A) (anti-SV40 poly(A) signal shRNA (SEQ ID NO: 161)) and the HIV poly(A) (anti-HIV poly(A) signal shRNA (SEQ ID NO: 162)), respectively. The shRNA expressed by pSO-Luc targets the Luciferase (luc) ORF. FIG. 2B shows a schematic presentation of the plasmids, psiCHECK2 and pHR′CMV-Luc, expressing the luc gene controlled by SV40 and HIV 1 poly(A) signals, respectively. Plasmid pGL3 was used as a target for siRNA directed against the luc ORF (anti-Luc ORF shRNA (SEQ ID NO: 163)). Bold letters above the lines indicates the target sequences. SV40-pr (SV40 promoter), R-Luc (Renilla Luciferase), syn pA (synthetic poly(A) TK pr (thymidine kinase promoter), h-Luc (humanized Luciferase), SV40 pA (SV40 poly(A)), CMV-pr (CMV promoter), LTR (HIV long terminal repeat), HIV LTR pA (the poly(A) located in the HIV LTRs).

FIGS. 3A-3B are graphs showing mediated reduction of Luciferase expression from vectors containing poly(A) signal sites of HIV and SV40. FIG. 3A shows Luciferase activity (RLU, relative light units) in HeLa cells transfected with increasing amounts of shRNA producing vectors: pSA-SV (▪), pSO-Luc (♦) and pSA-HIV as a specificity control, (x) or of 293T transfected with pSA-SV () and pSO-Luc (▴) are presented. As a target for siRNA activities expressed by either pSA-SV or pSA-HIV the cells were cotransfected with psiCHECK2. Plasmid pGL3 served as a target for siRNA made by pSO-LUC. Luciferase activity in the absence of siRNA was set as 100%. Luciferase (Firefly) activity was normalized to Renilla Luciferase activity in each transfection. FIG. 3B shows Luciferase activity (RLU, relative light units) in HeLa cells transfected with increasing amounts of pSA-HIV(♦), pSA-SV, as a specificity control(▪). 293T cells were transfected with increasing amounts of pSA-HIV(▴). As a target for the siRNA the cells were co-transfected with pHR′CMV-Luc.

FIGS. 4A-4B show the inhibition of lentiviral mRNA by siRNA targeting the HIV poly(A) signal. FIG. 4A shows Northern blot analysis of luc mRNA expressed from the lentiviral vector pHR′CMV-Luc in HeLa cells. Cells were transfected with pHR′CMV-Luc (PHR-Luc)- and cotransfected with either siRNA expressing vectors pSA-HIV, or pSO-Luc. The positions of the 28S and 18S RNA are indicated. FIG. 4B shows a quantitative illustration of the intensity of the bands monitored by Phospho-imager, (Fuji) and normalized to that of Beta-actin.

FIGS. 5A-5B show SiRNA mediated inhibition of SV40 late protein and viral propagation. FIG. 5A shows Western blot analysis of the SV40 VP1 protein in CV1 cells. Cells were cotransfected with SV40 DNA and with either pSO-Luc (Sh RNA against ORF of luc, SV40), or pSA-SV (SV40+pSA-SV). FIG. 5B shows quantification of VP1, the X ray film (see A) was scanned and the intensity of the bands (empty columns) was determined (see Materials and Methods). Viruses were harvested from the CV1 cells cotransfected with SV40 DNA and pSO-Luc (Control), or pSA-SV (siRNA) and the titer was determined 48 h following infection of CMT4 cells by in-situ hybridization to a specific SV40 DNA probe (full columns).

FIG. 6 shows the specific inhibition of ectopic CREB gene expression. The expression of CREB in C4 cells (diamonds) or stably transfected with vectors expressing either the ectopic wild type CREB (squares) or the dominant positive CREB300/310 (triangles) was determined following transient transfection, by a reporter vector pGLCRE-Hyg (diamonds). In this vector the luciferase gene is controlled by the CRE consensus sequence and the bovine growth hormone poly(A). The two CREB variants are controlled by the SV40 poly(A) signal. The vector pSA-SV expressing siRNA targeting SV-40 Poly(A) was cotransfected with the reporter plasmid at the concentrations indicated at the X axis. The results were normalized to the Renilla luciferase activity expressed from pBABE renilla vector (normalized RLU). The levels of luciferase activity induced by the endogenous native C4 encoded CREB (diamonds) are indicated.

### DETAILED DESCRIPTION OF THE INVENTION

The present invention provides methods for designing a sequence for efficient short interference RNA molecules (siRNA) directed to the consensus sequence of the polyadenylation signal site, in conjunction with unique sequences that mediates efficient and specific inhibition of gene expression in a dose dependent manner. The results of the present invention indicate that targeting the poly(A) site abrogates gene expression as effectively as targeting a sensitive internal coding sequence.

### DEFINITIONS

As used herein, the term “vector” refers to the plasmid, virus or phage chromosome used in cloning to carry the cloned DNA segment. Vectors capable of directing the expression of genes to which they are operatively linked are referred to herein as “expression vectors”. Another type of vector is a genomic integrated vector, or “integrated vector”, which can become integrated into the chromosomal DNA of the host cell. Another type of vector is an episomal vector, i.e., a nucleic acid capable of extra-chromosomal expression. In the present specification, “plasmid” and “vector” are used interchangeably unless otherwise clear from the context.

As used herein, the term “nucleic acid” refers to polynucleotides such as deoxyribonucleic acid (DNA), and, where appropriate, ribonucleic acid (RNA). The term should also be understood to include, as applicable to the embodiment being described, single-stranded (such as sense or antisense) and double-stranded polynucleotides.

As used herein, the term “gene” or “recombinant gene” refers to a nucleic acid comprising an open reading frame encoding a polypeptide of the present invention, including both exon and (optionally) intron sequences. A “recombinant gene” refers to nucleic acids encoding such regulatory polypeptides that may optionally include intron sequences that are derived from chromosomal DNA. The term “intron” refers to a DNA sequence present in a given gene that is not present in the mature RNA and is generally found between exons.

As used herein, “cell” refers to a eukaryotic cell. Typically, the cell is of animal origin and can be a stem cell or somatic cells. Suitable cells can be of, for example, mammalian, avian or plant origin. Examples of mammalian cells include human, bovine, ovine, porcine, murine, and rabbit cells. The cell can be an embryonic cell, bone marrow stem cell or other progenitor cell. Where the cell is a somatic cell, the cell can be, for example, an epithelial cell, fibroblast, smooth muscle cell, blood cell (including a hematopoietic cell, red blood cell, T-cell, B-cell, etc.), tumor cell, cardiac muscle cell, macrophage, dendritic cell, neuronal cell (e.g., a glial cell or astrocyte), or pathogen-infected cell (e.g., those infected by bacteria, viruses, virusoids, parasites, or prions).

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene.

As used herein, the terms “RNA” and “RNA molecule(s)” are used interchangeably to refer to RNA that mediates RNA interference. These terms include double-stranded RNA, single-stranded RNA, isolated RNA (partially purified RNA, essentially pure RNA, synthetic RNA, recombinantly produced RNA etc.), as well as altered RNA that differs from naturally occurring RNA by the addition, deletion, substitution and/or alteration of one or more nucleotides.

The term “loss-of-function”, as it refers to genes inhibited by the RNAi method of the present invention, refers to diminishment in the level of expression of a gene when compared to the level in the absence of the dsRNA constructs.

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence.

By “inhibit” it is meant that the activity of a gene expression product or level of RNAs or equivalent RNAs encoding one or more gene products is reduced below that observed in the absence of the nucleic acid molecule of the invention.

The term “silencing” as used herein refers to suppression of expression of the (target) gene. It does not necessarily imply reduction of transcription, because gene silencing is believed to operate in at least some cases post-transcriptionally. The degree of gene silencing can be complete so as to abolish production of the encoded gene product (yielding a null phenotype), but more generally the gene expression is partially silenced, with some degree of expression remaining (yielding an intermediate phenotype). The term should not therefore be taken to require complete “silencing” of expression.

As used herein, “introducing” refers to the transfer of a nucleic acid molecule from outside a host cell to inside a host cell. Nucleic acid molecules can be “introduced” into a host cell by any means known to those of skill in the art, for example as taught by Sambrook et al. Molecular Cloning: A Laboratory Manual, Cold Spring Harbor Laboratory Press, New York (2001), the contents of which are incorporated by reference herein. Means of “introducing” nucleic acids into a host cell include, but are not limited to heat shock, calcium phosphate transfection, electroporation, lipofection, and viral-mediated gene transfer.

As used herein, the term “transfection” refers to the introduction of a nucleic acid, e.g., an expression vector, into a recipient cell by nucleic acid-mediated gene transfer. “Transformation” as used herein, refers to a process in which a cell's genotype is changed as a result of the cellular uptake of exogenous DNA or RNA, and, for example, the transformed cell expresses a dsRNA construct.

As used herein, the term “infection” means the introduction of a nucleic acid by a virus into a recipient cell or organism. Viral infection of a host cell is a technique which is well established in the art and can be found in a number of laboratory texts and manuals such as Sambrook et al., Molecular Cloning: A Laboratory Manual, Vol. 1-3, Cold Spring Harbor Laboratory, Cold Spring Harbor, N.Y., 2001.

### Effective Gene Silencing

The present invention provides methods for attenuating or inhibiting gene expression in a cell using gene-targeted double stranded RNA (dsRNA). The dsRNA contains a nucleotide sequence that hybridizes under physiologic conditions of the cell to the nucleotide sequence of at least a portion of the target mRNA of the gene to be inhibited (target gene).

The polyadenylation signal site of eukaryotic mRNAs commonly comprises 6 bases that are located 10-30 bases upstream of the poly(A) tail. The siRNAs of the present invention will typically comprise 15-40 nucleotides comprising at least two parts, a first part comprising a consensus sequence corresponding to at least a part of the polyadenylation signal site and a second part comprising unique sequence corresponding to 9-34 contiguous or non-contiguous nucleotides from the region adjacent to said polyadenylation signal. The unique sequences adjacent to the consensus polyadenylation signal can be on the 3′ side, on the 5′ side or both.

It should be appreciated that the present invention also encompasses Poly(A) signal sites that comprise a shorter or longer number of nucleotides.

According to current knowledge, in 68% of the human genes, the 6 nucleotides AAUAAA of the consensus sequence of the Poly(A) signal are flanked by unique sequences of at least 15 nucleotides. Most of the remaining genes (32%) include multi-copy genes or mRNA splice variants of the same gene.

The method described herein does not require 100% sequence identity between the siRNA and the target gene. By utilizing bio-informatic tools, the sequence can contain mismatch pairs of nucleotides. Thus, the methods of the invention have the advantage of being able to tolerate some sequence variations that might be expected due to genetic mutation, strain polymorphism, or evolutionary divergence.

In order to establish that in accordance with the principles of the present invention the polyadenylation consensus signal poly(A) can serve as a general target and yet as unique gene specific sequences for siRNA activity, the inventors of the present invention used a pSilencer expression vector which comprises the human U6 promoter known to express siRNAs in mammalian cells (Ambion Corp) and the siRNA homologous to the consensus sequence AAUAAA in conjugation with non coding sequences.

The target gene can be a gene derived from the cell (i.e., a cellular gene), an endogenous gene (i.e., a cellular gene present in the genome), a transgene (i.e., a gene construct inserted at an ectopic site in the genome of the cell), or a gene from a pathogen (such as a virus, bacterium, fungus or protozoan) which is capable of infecting an organism. Depending on the particular target gene and the dose of double stranded RNA material delivered, this process can provide partial or complete loss of function for the target gene.

Inhibition of gene expression refers to the absence (or observable decrease) in the level of protein and/or mRNA product from a target gene. Specificity refers to the ability to inhibit the target gene without manifesting effects on other genes of the cell. According to the present invention, quantification of the amount of gene expression allows one to determine the degree of inhibition which is greater than 50%, preferably 65%, more preferably 75%, and most preferably 95% and more.

As exemplified hereinbelow, the two siRNA expression vectors, one targeted to the HIV-LTR polyadenylation signal sequence and the other targeted to the SV40 late polyadenylation signal sequence, inhibited, in a dose dependent manner, Luciferase activity. The efficiency of silencing by the siRNA directed against the poly(A) signal was compared to that of the siRNA directed to a protein coding sequence of the Luciferase mRNA. This internal sequence has previously been shown to be very sensitive to siRNA inhibition (Elbashir, S M et al., Nature 411: 494-498, 2001). The results of the present invention indicate that targeting the poly(A) site abrogates gene expression as effectively as targeting a known sensitive internal coding sequence.

Designing siRNAs According to the Invention

Computational analysis demonstrated a high conservation of the poly(A) signal of both cell and viral mRNAs. The inventors of the present invention found that 97.45% of human mRNA 3′ UTRs harbor an AAUAAA sequence, which is flanked by unique sequences of at least 15 bases. The remaining 3′UTRs, that have redundant poly(A) regions, include poly(A) regions, that are shared among several genome locations, but are annotated to be producing the same protein. Many of the others belong to different genes that produce different proteins, but belong to the same protein family.

Exemplary siRNAs based on the human mRNA 3′UTR sequences of a broad range of gene functions designed according to the principles of the present invention are presented in Table 1. The inhibition of the exemplary gene by the siRNA will typically reduce the phenotypic expression of the gene of interest in eukaryotic cells. However, besides the expected loss of function phenotype, previously unknown functions or phenotypes may become apparent upon gene silencing. It will be appreciated by the skilled artisan that siRNA may be used to decipher gene pathways and interactions or to confirm interactions.

TABLE 1
EXEMPLARY SIRNAS OF THE PRESENT INVENTION

| | | Reference | | | |
|---|---|---|---|---|---|
| | | Sequence | | | SEQ |
| Gene | | Database | | | ID |
| Symbol | Gene Product | No. | Gene function | siRNA sequences | NO. |
| ADH1A | Alcohol | NM_000667 | Alcohol | sense; | 1 |
| | dehydrogenase | | metabolism | 5′-aattgaagccaataaaccttcc | |
| | 1A (class I), alpha | | | anti-sense; | 2 |
| | polypeptide | | | 5′-ggaaggtttattggcttcaatt; | |
| AQP4 | Aquaporin 4 | NM_001650 | Neurogenesis | sense; | 3 |
| | | /// | | 5′-tgtgaaaataaacatttggatg | |
| | | NM_004028 | | anti-sense; | 4 |
| | | | | 5′-catccaaatgtttattttcaca; | |
| ARHGAP1 | Rho GTPase | NM_004308 | Rho protein | sense; | 5 |
| | activating protein 1 | | signal | 5′-gtatttcaataaaaatgttga; | |
| | | | transduction | anti-sense; | 6 |
| | | | | 5′-tcaacatttttattgaaatac; | |
| ATR | Ataxia | NM_001184 | Cell cycle | sense; | 7 |
| | telangiectasia and | | checkpoint | 5′-cagttattaagaaataaactgc | |
| | Rad3 related | | | anti-sense; | 8 |
| | | | | 5′-gcagtttatttcttaataactg; | |
| BCL2L2 | BCL2-like 2 | NM_004050 | anti-apoptosis | sense; | 9 |
| | | | | 5′-aataaataaagcccagaagttt | |
| | | | | anti-sense; | 10 |
| | | | | 5′-aaacttctgggctttatttatt; | |
| CD4 | CD4 antigen | NM_000616 | Cell | sense; | 11 |
| | (p55) /// CD4 | | differentiation | 5′-gctcccctgagctgaaataaa; | |
| | antigen (p55) | | | anti-sense; | 12 |
| | | | | 5′-tttatttcagctcaggggagc; | |
| CDK5R1 | Cyclin-dependent | NM_003885 | Regulation of | sense; | 13 |
| | kinase 5, | | cyclin | 5′-gtactgtgctgattcaataaa; | |
| | regulatory subunit | | dependent | anti-sense; | 14 |
| | 1 (p35) | | protein kinase | 5′-tttattgaatcagcacagtac; | |
| | | | activity | | |
| CEBPB | CCAAT/enhancer | NM_005194 | Regulation of | sense; | 15 |
| | binding protein | | transcription | 5′-aagaacacttttaataaac; | |
| | (C/EBP), beta | | | anti-sense; | 16 |
| | | | | 5′-gtttattaaaagtgttctt; | |
| CHRM2 | Cholinergic | NM_000739 | G-protein | sense; | 17 |
| | receptor, | /// | signaling, | 5′-cttcaaatagtggcaaataaa; | |
| | muscarinic 2 | NM_001006 | coupled to | anti-sense; | 18 |
| | | 626 /// | cAMP | 5′-tttatttgccactatttgaag; | |
| | | NM_001006 | ucleotide | | |
| | | 627 /// | second | | |
| | | NM_001006 | messenger | | |
| | | 628 /// | | | |
| | | NM_001006 | | | |
| | | 629 /// | | | |
| | | NM_001006 | | | |
| | | 630 /// | | | |
| | | NM_001006 | | | |
| | | 631 /// | | | |
| | | NM_001006 | | | |
| | | 632 /// | | | |
| | | NM_001006 | | | |
| | | 633 | | | |
| CHRM5 | Cholinergic | NM_012125 | Acetylcholine | sense; | 19 |
| | receptor | | receptor | 5′ttctactaataaagatggatcaa | |
| | muscarinic 5 | | signaling, | anti-sense; | 20 |
| | | | muscarinic | 5′-ttgatccatctttattagtagaa | |
| | | | pathway | | |
| COX5A | Cytochrome c | NM_004255 | Electron | sense; | 21 |
| | oxidase subunit | | transport | 5′-cttggactttaataaaaggga; | |
| | Va | | | anti-sense; | 22 |
| | | | | 5′-tcccttttattaaagtccaag; | |
| COX8A | Cytochrome c | NM_004074 | Electron | sense; | 23 |
| | oxidase subunit | | transport | 5′-cccttgtaacaataaaatcta; | |
| | 8A (ubiquitous) | | | anti-sense; | 24 |
| | | | | 5′-tagattttattgttacaaggg; | |
| CTSF | Cathepsin F | NM_003793 | Proteolysis and | sense; | 25 |
| | | | peptidolysis | 5′-acagcaataaagaggtgtcct; | |
| | | | | anti-sense; | 26 |
| | | | | 5′-aggacacctctttattgctgt; | |
| CXCR3 | Chemokine (C-X- | NM_001504 | Antimicrobial | sense; | 27 |
| | C motif) receptor 3 | | humoral | 5′ttcaataaacaagatcgtcagg; | |
| | | | response | anti-sense; | 28 |
| | | | | 5′-cctgacgatcttgtttattgaa; | |
| CXCR6 | Chemokine (C-X- | NM_006564 | Viral genome | sense; | 29 |
| | C motif) receptor 6 | | replication | 5′-gtttcatagctaagaaataaa; | |
| | | | | anti-sense; | 30 |
| | | | | 5′-tttatttcttagctatgaaac; | |
| CYC1 | Cytochrome c-1 | NM_001916 | Electron | sense; | 31 |
| | | | transport | 5′-catcatgggaataaattaatt; | |
| | | | | anti-sense; | 32 |
| | | | | 5′-aattaatttattcccatgatg; | |
| CYCS | Cytochrome c, | NM_018947 | Caspase | sense; | 33 |
| | somatic | | activation | 5′-gtttagtgtgtatcaataaa; | |
| | | | | anti-sense; | 34 |
| | | | | 5′-tttattgatacacactaaac; | |
| CYP11B2 | Cytochrome | NM_000498 | C21-steroid | sense; | 35 |
| | P450, family 11, | | hormone | 5′- | |
| | subfamily B, | | biosynthesis | gatcctaaaataaaccttggaa; | |
| | polypeptide 2 | | | anti-sense; | 36 |
| | | | | 5′-ttccaaggtttattttaggatc; | |
| DES | Desmin | NM_001927 | Cytoskeleton | sense; | 37 |
| | | | organization | 5′- | |
| | | | and biogenesis | gctctggagagaaacaataaa; | |
| | | | | anti-sense; | 38 |
| | | | | 5′-tttattgtttctctccagagc; | |
| DIPA | Hepatitis delta | NM_006848 | Regulates early | sense; | 39 |
| | antigen- | | events of | 5′- | |
| | interacting protein A | | adipogenesis | taataaacccggacggaagcg; | |
| | | | | anti-sense; | 40 |
| | | | | 5′-cgcttccgtccgggtttatta; | |
| ECEL1 | Endothelin | NM_004826 | Proteolysis and | sense; | 41 |
| | converting | | peptidolysis | 5′-ctgca aagtctggtc | |
| | enzyme-like 1 | | | aataaa; | |
| | | | | anti-sense; | 42 |
| | | | | 5′-tttattgaccagactttgcag; | |
| EIF2AK3 | Eukaryotic | NM_004836 | Coordinating | sense; | 43 |
| | translation | | stress gene | 5′-caagtctaaatgatttaataaa; | |
| | initiation factor 2- | | responses | anti-sense; | 44 |
| | alpha kinase 3 | | | 5′-tttattaaatcatttagacttg; | |
| eIF3k | Eukaryotic | NM_013234 | Protein | sense; | 45 |
| | translation | | biosynthesis | 5′-cttcaggtgtttaataaagat; | |
| | initiation factor 3 | | | anti-sense; | 46 |
| | subunit k | | | 5′-atctttattaaacacctgaag; | |
| EIF4EBP1 | Eukaryotic | NM_004095 | Negative | sense; | 47 |
| | translation | | regulation of | 5′- | |
| | initiation factor | | protein | caagagaggaaataaaagcca; | |
| | 4E binding | | biosynthesis | anti-sense; | 48 |
| | protein 1 | | | 5′-tggcttttatttcctctcttg; | |
| EPHB3 | EPH receptor B3 | NM_004443 | Transmembrane | sense; | 49 |
| | | | receptor protein | 5′- | |
| | | | tyrosine kinase | ctgggccgacagcagaataaa; | |
| | | | signaling | anti-sense; | 50 |
| | | | pathway | 5′-tttattctgctgtcggcccag; | |
| F2 | Coagulation | NM_000506 | STAT protein | sense; | 51 |
| | factor II | | nuclear | 5′-aactatggttcccaataaaag; | |
| | (thrombin) | | translocation | anti-sense; | 52 |
| | | | | 5′-cttttattgggaaccatagtt; | |
| FOSL1 | FOS-like antigen 1 | NM_005438 | Cellular defense | sense; | 53 |
| | | | response | 5′-caaaatggaataaaaggcttg; | |
| | | | | anti-sense; | 54 |
| | | | | 5′-caagccttttattccattttg; | |
| FUT9 | Fucosyltransferase | NM_006581 | L-fucose | sense; | 55 |
| | 9 (alpha (1,3) | | catabolism | 5′-atagaaccaaataaacctacc; | |
| | fucosyltransferase) | | | anti-sense; | 56 |
| | | | | 5′-ggtaggtttatttggttctat; | |
| GABRD | Gamma- | NM_000815 | Ion transport | sense; | 57 |
| | aminobutyric acid | | | 5′- | |
| | (GABA) A | | | ctggtcccagcatgaaataaag; | |
| | receptor, delta | | | anti-sense; | 58 |
| | | | | 5′-ctttatttcatgctgggaccag; | |
| GDF3 | Growth | NM_020634 | Cell growth and | sense; | 59 |
| | differentiation | | maintenance | 5′-ttaataaaactacctatctgg; | |
| | factor 3 | | | anti-sense; | 60 |
| | | | | 5′-ccagataggtagttttattaa; | |
| GPR35 | G protein-coupled | NM_005301 | G-protein coupled | sense; | 61 |
| | receptor 35 | | receptor protein | 5′- | |
| | | | signaling pathway | cccctcggggctggaataaaa; | |
| | | | | anti-sense; | 62 |
| | | | | 5′-ttttattccagccccgagggg; | |
| GPR4 | G protein-coupled | NM_005282 | G-protein coupled | sense; | 63 |
| | receptor 4 | | receptor protein | 5′-caccatacacaagtaaataaa; | |
| | | | signaling pathway | anti-sense; | 64 |
| | | | | 5′-tttatttacttgtgtatggtg; | |
| GSTA3 | Glutathione S- | NM_000847 | Response to stress | sense; | 65 |
| | transferase A3 | | | 5′-aataaaaactcctatttgcta; | |
| | | | | anti-sense; | 66 |
| | | | | 5′-tagcaaataggagtttttatt; | |
| GSTT1 | Glutathione S- | NM_000853 | Response to stress | sense; | 67 |
| | transferase theta 1 | | | 5′-ttggataataaacctggctca; | |
| | | | | anti-sense; | 68 |
| | | | | 5′-tgagccaggtttattatccaa; | |
| HDAC3 | Histone | NM_003883 | Regulation of cell | sense; | 69 |
| | deacetylase 3 | | cycle | 5′-tatccaataaactaagtcggt; | |
| | | | | anti-sense; | 70 |
| | | | | 5′-accgacttagtttattggata; | |
| HEAB | ATP/GTP- | NM_006831 | mRNA | sense; | 71 |
| | binding protein | | processing | 5′-agagggactccttccaataaa; | |
| | | | | anti-sense; | 72 |
| | | | | 5′-ttattggaaggagtccctct; | |
| HEBP1 | Heme binding | NM_015987 | Circadian rhythm | sense; | 73 |
| | protein 1 | | | 5′-aataaaaggcattgacttaaa; | |
| | | | | anti-sense; | 74 |
| | | | | 5′-tttaagtcaatgccttttatt; | |
| HOXC5 | Homeo box C5 | NM_018953 | Regulation of | sense; | 75 |
| | | | transcription from | 5′-tgtcatatcaaataaagagag; | |
| | | | Pol II promoter | anti-sense; | 76 |
| | | | | 5′-ctctctttattttgatatgaca; | |
| HRASLS3 | HRAS-like | NM_007069 | Associated with | sense; | 77 |
| | suppressor 3 | | tumor inhibitory | 5′- | |
| | | | activities | ttcacagaataaaataaagcaa; | |
| | | | | anti-sense; | 78 |
| | | | | 5′-ttgcttttattttattctgtgaa; | |
| HSPA6 | Heat shock | NM_002155 | Protein folding | sense; | 79 |
| | 70 kDa protein 6 | | | 5′-atagttatagacctaaataaa; | |
| | (HSP70B′) | | | anti-sense; | 80 |
| | | | | 5′-tttatttaggtctataactat; | |
| HSPB7 | Heat shock | NM_014424 | Protein folding | sense; | 81 |
| | 27 kDa protein | | | 5′-ggaacctgtatacacaataaa; | |
| | family, member 7 | | | anti-sense; | 82 |
| | (cardiovascular) | | | 5′-tttattgtgtatacaggttcc; | |
| INSL5 | Insulin-like 5 | NM_005478 | L-fucose | sense; | 83 |
| | | | catabolism | 5′-gctgcgcaaaattgcaataaa; | |
| | | | | anti-sense; | 84 |
| | | | | 5′-tttattgcaattttgcgcagc; | |
| INSM1 | Insulinoma- | NM_002196 | Regulation of | sense; | 85 |
| | associated 1 | | transcription | 5′-caaataaaatattttcaaagtc; | |
| | | | | anti-sense; | 86 |
| | | | | 5′-gactttgaaaatattttatttg; | |
| IRS2 | Insulin receptor | NM_003749 | Glucose | sense; | 87 |
| | substrate 2 | | metabolism | 5′-agccatatgcaataaaataaa; | |
| | | | | anti-sense; | 88 |
| | | | | 5′-tttattttattgcatatggct; | |
| K-RAS | | M54968/// | Transducing | sense; | 89 |
| | | M38506 | growth-promoting | 5′- | |
| | | | signals | aaggaataaacttgattatattg; | |
| | | | | anti-sense; | 90 |
| | | | | 5′-caatataatcaagtttattcctt; | |
| LPL | Lipoprotein lipase | NM_000237 | Fatty acid | sense; | 91 |
| | | | metabolism | 5′-atggaatcagcttttaataaa; | |
| | | | | anti-sense; | 92 |
| | | | | 5′-tttattaaaagctgattccat; | |
| LTA | Lymphotoxin | NM_000595 | Induction of | sense; | 93 |
| | alpha (TNF | | apoptosis | 5′-ccctcgatgaagcccaataaa; | |
| | superfamily, | | | anti-sense; | 94 |
| | member 1) | | | 5′-tttattgggcttcatcgaggg; | |
| MAGEA5 | Melanoma | NM_021049 | Cancer-specific | sense; | 95 |
| | antigen, family A, 5 | | antigen | 5′-gacaaattaaatctgaataaa; | |
| | | | | anti-sense; | 96 |
| | | | | 5′-tttattcagatttaatttgtc; | |
| MAP3K11 | Mitogen-activated | NM_002419 | Protein amino | sense; | 97 |
| | protein kinase 11 | | acid | 5′- | |
| | | | phosphorylation | gtgaagccagaagccaaataaa; | |
| | | | | anti-sense; | 98 |
| | | | | 5′-tttatttggcttctggcttcac; | |
| MYC | V-myc | NM_002467 | Cell proliferation | sense; | 99 |
| | myelocytomatosis | | | 5′-aataaaataactggcaaatat; | |
| | viral oncogene | | | anti-sense; | 100 |
| | homolog (avian) | | | 5′-atatttgccagttattttatt; | |
| MYD88 | Myeloid | NM_002468 | Regulation of I- | sense; | 101 |
| | differentiation | | kappaB | 5′- | |
| | primary response | | kinase/NF- | gcatcctgagtttataataataaa; | |
| | gene (88) | | kappaB cascade | anti-sense; | 102 |
| | | | | 5-′tttattattataaactcaggatgc | |
| OAZ2 | Ornithine | NM_002537 | Polyamine | sense; | 103 |
| | decarboxylase | | metabolism | 5′-ttgtgttactgtgtcaataaa; | |
| | antizyme 2 | | | anti-sense; | 104 |
| | | | | 5′-tttattgacacagtaacacaa; | |
| OSR1 | Oxidative-stress | NM_005109 | Response to | sense; | 105 |
| | responsive 1 | | oxidative stress | 5′-atcaataaagagtaaattgtc; | |
| | | | | anti-sense; | 106 |
| | | | | 5′-gacaatttactctttattgat; | |
| PAH | Phenylalanine | NM_000277 | Phenylalanine | sense; | 107 |
| | hydroxylase | | catabolism | 5′-ttagtaataaaacattagtag; | |
| | | | | anti-sense; | 108 |
| | | | | 5′-ctactaatgttttattactaa; | |
| POLR2A | Polymerase | NM_000937 | Transcription | sense; | 109 |
| | (RNA) II (DNA | | from Pol II | 5′-tgaagtttaaataaagtttac; | |
| | directed) | | promoter | anti-sense; | 110 |
| | polypeptide A, | | | 5′-gtaaactttatttaaacttca; | |
| | 220 kDa | | | | |
| PPIH | Peptidyl prolyl | NM_006347 | Nuclear mRNA | sense; | 111 |
| | isomerase H | | splicing | 5′-ttcaactgtaaataaagttt; | |
| | (cyclophilin H) | | | anti-sense; | 112 |
| | | | | 5′-aaactttatttacagttgaa; | |
| PRKCE | Protein kinase C, | NM_005400 | Induction of | sense; | 113 |
| | epsilon | | apoptosis | 5′-attgtttcagaacctaataaa; | |
| | | | | anti-sense; | 114 |
| | | | | 5′-tttattaggttctgaaacaat; | |
| PRKRA | Protein kinase, | NM_003690 | Negative | sense; | 115 |
| | interferon- | | regulation of cell | 5′- | |
| | inducible double | | proliferation | gaaattcaaaggtgaaaataaa; | |
| | stranded RNA | | | anti-sense; | 116 |
| | dependent | | | 5′-tttattttcacctttgaatttc; | |
| | activator | | | | |
| PRND | Prion protein 2 | NM_012409 | Participate in the | sense; | 117 |
| | (doublet) | | glial response | 5′-tttgccactgcaaacaataaa; | |
| | | | around amyloid | anti-sense; | 118 |
| | | | cores | 5′-tttattgtttgcagtggcaaa; | |
| PSMD1 | Proteasome | NM_002807 | Regulation of cell | sense; | 119 |
| | (prosome, | | cycle | 5′-caaataaatataagatctccag; | |
| | macropain) 26S | | | anti-sense; | 120 |
| | subunit, non- | | | 5′-ctggagatcttatatttatttg; | |
| | ATPase, 1 | | | | |
| PTTG1IP | Pituitary tumor- | NM_004339 | Protein-nucleus | sense; | 121 |
| | transforming 1 | | import | 5′-aaccagtttccaataaaacgg; | |
| | interacting protein | | | anti-sense; | 122 |
| | | | | 5′-ccgttttattggaaactggtt; | |
| QARS | Glutaminyl-tRNA | NM_005051 | Organization of | sense; | 123 |
| | synthetase | | the mammalian | 5′-cccaaattccatgtcaataaa; | |
| | | | multienzyme | anti-sense; | 124 |
| | | | synthetase | 5′-tttattgacatggaatttggg; | |
| | | | complex | | |
| RAD23A | RAD23 homolog | NM_005053 | Nucleotide- | sense; | 125 |
| | A (S. cerevisiae) | | excision repair | 5′-aaaggttttgaagtgaataaa; | |
| | | | | anti-sense; | 126 |
| | | | | 5′-tttattcacttcaaaaccttt; | |
| RHOG | Ras homolog | NM_001665 | Regulation of cell | sense; | 127 |
| | gene family, | | proliferation | 5′ccatcagcatcaataaaacctc; | |
| | member G (rho | | | anti-sense; | 128 |
| | G) | | | 5′-gaggttttattgatgctgatgg; | |
| RIN1 | Ras and Rab | NM_004292 | Intracellular | sense; | 129 |
| | interactor 1 | | signaling cascade | 5′-catctgaggaactggaataaa; | |
| | | | | anti-sense; | 130 |
| | | | | 5′-tttattccagttcctcagatg; | |
| RNASEH1 | Ribonuclease H1 | NM_002936 | RNA catabolism | sense.; | 131 |
| | | | | 5′agaccaagaagcataaataaa | |
| | | | | anti-sense; | 132 |
| | | | | 5′-tttatttatgcttcttggtct; | |
| STAT5B | Signal transducer | NM_012448 | JAK-STAT | sense; | 133 |
| | and activator of | | cascade | 5′-atgttacaataaagccttcct; | |
| | transcription 5B | | | anti-sense; | 134 |
| | | | | 5′-aggaaggctttattgtaacat; | |
| TBCA | Tubulin-specific | NM_004607 | Tubulin-folding | sense; | 135 |
| | chaperone A | | | 5′-tgtcaaataaatgagttcatc; | |
| | | | | anti-sense; | 136 |
| | | | | 5′-gatgaactcatttatttgaca; | |
| THBS2 | Thrombospondin 2 | NM_003247 | Cell adhesion | sense; | 137 |
| | | | | 5′-aagattaacaacaggaaataa | |
| | | | | a; | |
| | | | | anti-sense; | 138 |
| | | | | 5′-tttatttcctgttgttaatctt; | |
| TNFRSF10D | Tumor necrosis | NM_003840 | Apoptosis | sense; | 139 |
| | factor receptor | | | 5′-aataaatatgaaacctcatat; | |
| | superfamily, | | | anti-sense; | 140 |
| | member 10d, | | | 5′-atatgaggtttcatatttatt; | |
| | decoy with | | | | |
| | truncated death | | | | |
| | domain | | | | |
| UBE2N | Ubiquitin- | NM_003348 | Ubiquitin cycle | sense; | 141 |
| | conjugating | | | 5′-ctggtatccttccaaataaa; | |
| | enzyme E2N | | | anti-sense; | 142 |
| | (UBC13 | | | 5′-tttatttggaaggataccag; | |
| | homolog, yeast) | | | | |
| UQCRH | Ubiquinol- | NM_006004 | Aerobic | sense; | 143 |
| | cytochrome c | | respiration | 5′cgcaatgattccatctaaataaa | |
| | reductase hinge | | | anti-sense; | 144 |
| | protein | | | 5′tttatttagatggaatcattgcg; | |
| USP18 | Ubiquitin specific | NM_017414 | Ubiquitin cycle | sense; | 145 |
| | protease 18 | | | 5′-aacacagtcatgaataaagtt; | |
| | | | | anti-sense; | 146 |
| | | | | 5′-aactttattcatgactgtgtt; | |
| UTF1 | Undifferentiated | NM_003577 | Regulation of | sense; | 147 |
| | embryonic cell | | transcription from | 5′-ttcttgggtacgttcaataaa; | |
| | transcription | | Pol II promoter | anti-sense; | 148 |
| | factor 1 | | | 5′-tttattgaacgtacccaagaa; | |
| WAS | Wiskott-Aldrich | NM_000377 | Actin | sense; | 149 |
| | syndrome protein | | polymerization | 5′-aataaaagaattgtctttctgt; | |
| | eczema- | | and/or | anti-sense; | 150 |
| | thrombocytopenia | | depolymerization | 5′-acagaaagacaattcttttatt; | |
| WISP3 | WNT1 inducible | NM_198239 | Regulation of cell | sense; | 151 |
| | signaling pathway | | growth | 5′-aatcctgtcatataataaaaa; | |
| | protein 3 | | | anti-sense; | 152 |
| | | | | 5′-tttttattatatgacaggatt; | |
| ZNF24 | Zinc finger | NM_006965 | Regulation of | sense; | 153 |
| | protein 24 (KOX | | transcription | 5′-gcatacagtctaaataataaa; | |
| | 17) | | | anti-sense; | 154 |
| | | | | 5′-tttattatttagactgtatgc; | |

siRNAs Directed to Viral Poly(A) Signal Efficiently Inhibit Viral Replication.

One of the major problems associated with the application of the RNAi technology for virus inhibition is the rapid evolution of resistant escape mutants (Boden, D., et al., J. Virol. 77:11531-5, 2003). Usually the resistance viruses show silent mutations in the siRNA target sequence. The non-translated poly(A) signal and its flanking sequences are highly conserved in viruses and less tolerant to mutations. Thus, siRNA targeted against the poly(A) signal regions of viruses can efficiently inhibit viral gene expression and subsequent viral replication.

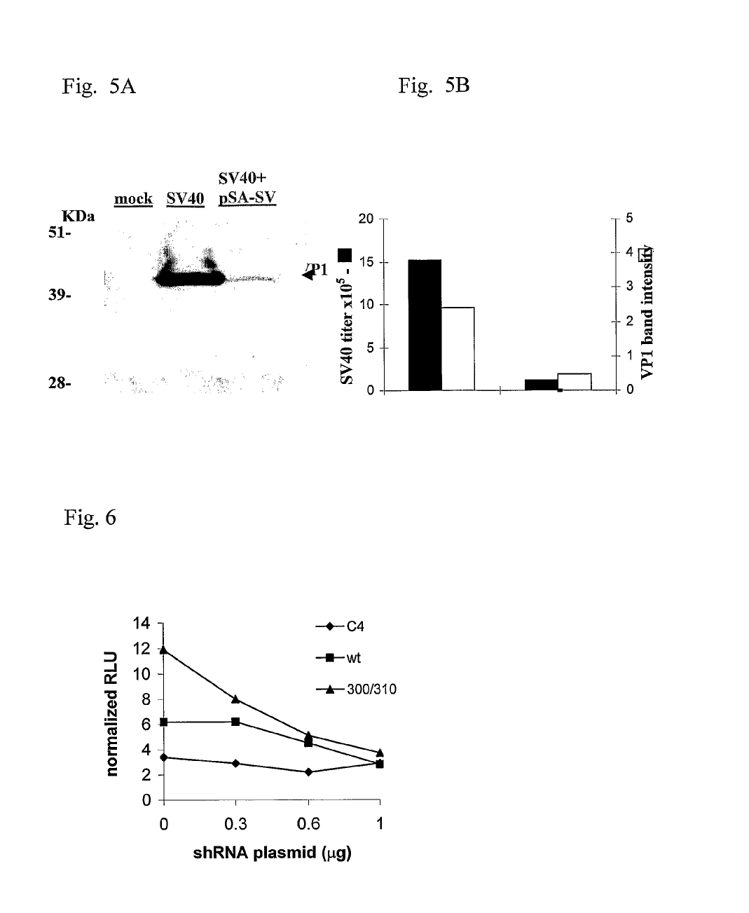

The genome of SV40, for instance, is a circular dsDNA transcribed from two promoters, controlling the expression of the early and late viral functions, wherein each of these two transcripts is regulated by a different poly(A) signal. siRNA-mediated inhibition of the SV40 late genes also affects SV40 viral propagation (see example 5 and FIG. 5B).

As described herein, cells were co-transfected with SV40 complete genome DNA and pSA-SV (targeting the SV40 late poly (A) region). 72 hrs following transfection, cell cultures were lysed, the proteins were resolved by PAGE and subjected to Western blot analysis, with antibodies specific for the SV40 VP1 capsid protein. In cells co-transfected with pSA-SV, the VP1 protein levels were 16 fold lower as compared with the control cells.

In addition, CMT4 cells were co-transfected with SV40 DNA and pSA-SV or with SV40 DNA and a non-relevant siRNA vector (pSO-Luc). At different time intervals after transfection, progeny virus was harvested, diluted and quantified by infection of CMT4 cells. Virus titers dropped dramatically (87%) in cells co-transfected with the pSA-SV, relative to the control transfection.

As indicated above, one aspect of the present invention provides methods of employing siRNA to modulate expression of a viral target gene or genes in a cell or organism including such a cell harboring a target viral genome. In further embodiments, the present invention provides methods of reducing viral gene expression of one or more target genes in a host cell. Reducing expression means that the level of expression of a target gene or coding sequence is reduced or inhibited by at least about 50%, usually by at least about 65%, preferably 75%, 80%, 85%, 90%, 95% or more, as compared to a control. Modulating expression of a target gene refers to reducing transcription and/or translation of a coding sequence, including genomic DNA, mRNA etc., into a polypeptide, or protein. In further embodiments, the present invention provides methods of reducing or inhibiting viral replication of one or more target genes in a host organism. Reducing replication means that the level of replication of a target viral genome is reduced or inhibited by at least about 2-fold, usually by at least about 5-fold, e.g., 10-fold, 15-fold, 20-fold, or more, as compared to a control. In certain embodiments, the replication of the target viral genome is reduced to such an extent that replication of the target viral genome is effectively inhibited.

Applications of siRNA

The present invention also relates to a variety of applications in which it is desired to modulate, e.g., one or more target genes, viral replication of a pathogenic virus, etc., in a whole eukaryotic organism, e.g., a mammal or a plant; or portion thereof, e.g., tissue, organ, cell, etc. In such methods, an effective amount of an RNAi active agent is administered to the host or introduced into the target cell. The term “effective amount” refers to a dosage sufficient to modulate expression of the target viral gene(s), as desired, e.g., to achieve the desired inhibition of viral replication. As indicated above, in certain embodiments of this type of application, the subject methods are employed to reduce expression of one or more target genes in the host in order to achieve a desired therapeutic outcome.

When the target gene is a viral gene, e.g., when inhibition of viral replication is desired, the target viral gene can be from a number of different viruses. Representative viruses include, but are not limited to: HBV, HCV, HIV, influenza A, Hepatitis A, picornaviruses, alpha-viruses, herpes viruses, and the like.

The methods described herein are also suitable for inhibiting the expression of a target gene in a tumor cell. The present invention relates to any type of cancer including solid tumors and non-solid tumors. The solid tumors are exemplified by CNS tumors, liver cancer, colorectal carcinoma, breast cancer, gastric cancer, pancreatic cancer, bladder carcinoma, cervical carcinoma, head and neck tumors, vulvar cancer and dermatological neoplasms including melanoma, squamous cell carcinoma and basal cell carcinomas. Non-solid tumors include lymphoproliferative disorders including leukemias and lymphomas.

Another application in which the subject methods find use is the elucidation of gene function by a functional analysis of eukaryotic cells, or eukaryotic non-human organisms, preferably mammalian cells or organisms and most preferably human cells, e.g. cell lines such as HeLa or 293, or rodents, e.g. rats and mice. By transfection with vector molecules which are homologous to a predetermined target gene encoding a suitable RNA molecule, a specific knockdown phenotype can be obtained in a target cell, e.g. in cell culture or in a target organism.

The present invention is also useful to produce plants with improved characteristics including but not limited to decreased susceptibility to climate injury, insect infestation, pathogen infection, and improved ripening characteristics. Any gene or genes that may be detrimental in the agricultural community could be a potential target or targets of such specially selected RNAs.

### Machinery of RNA Silencing Pathways

As described previously, RNAi phenomenon is mediated by a set of enzymatic activities, including an essential RNA component, that are evolutionarily conserved in eukaryotes ranging from plants to mammals. One enzyme contains an essential RNA component. After partial purification, a multi-component nuclease (herein “RISC nuclease”) co-fractionates with a discrete, 22-nucleotide RNA species which may confer specificity to the nuclease through homology to the substrate mRNAs. The short RNA molecules are generated by a processing reaction from the longer input dsRNA. Without wishing to be bound by any particular theory, these 22mer guide RNAs may serve as guide sequences that instruct the RISC nuclease to destroy specific mRNAs corresponding to the dsRNA sequences.

It has been demonstrated that short hairpin homologous to the 3′ UTR of genes, micro RNAs may also inhibit gene expression by a different mechanism than siRNAs, in most cases by stalling translation of the specific gene (Bartel, D P., Cell 23:116(2):281-297, 2004).

As exemplified hereinbelow, mRNA levels measured by band intensity normalized to β-actin mRNA from the same sample, were ten fold lower in cells co-transfected with a siRNA expressing plasmid. Without wishing to be bound to any one theory or mechanism of action, it appears that this type of gene silencing is mediated by specific degradation of mRNA involving an RNAi mechanism and not a microRNA mechanism which is less specific.

A preferred RNA-based method for generating loss of function phenotypes in putative interactor genes is by double-stranded RNA interference (dsRNAi) which has proven to be of great utility in genetic studies of C. elegans, and can also be used in Drosophila. In one approach, dsRNA can be generated by transcription in vivo.

International Patent Publication Nos. WO 99/32619 and WO 01/68836 suggest that RNA for use in siRNA can be chemically or enzymatically synthesized. The enzymatic synthesis contemplated is by a cellular RNA polymerase or a bacteriophage RNA polymerase (e.g., T3, T7, SP6) via the use and production of an expression construct as is known in the art (see for example, U.S. Pat. No. 5,795,715). The contemplated constructs provide templates that produce RNAs that contain nucleotide sequences identical to a portion of the target gene. The length of identical sequences provided by these references is at least 25 bases, and can be as many as 400 or more bases in length. An important aspect of this reference is that the inventors contemplate digesting longer dsRNAs to 21-25mer lengths with the endogenous nuclease complex that converts long dsRNAs to siRNAs in vivo. They do not, however, describe or present data for synthesizing and using in vitro transcribed 21-25mer dsRNAs. No distinction is made between the expected properties of chemical or enzymatically synthesized dsRNA in its use in RNA interference.

WO 01/12824 discloses methods and means for reducing the phenotypic expression of a nucleic acid of interest in eukaryotic cells, particularly in plant cells, by providing aberrant, possibly unpolyadenylated, target-specific RNA to the nucleus of the host cell. Unpolyadenylated target-specific RNA can be provided by transcription of a chimeric gene comprising a promoter, a DNA region encoding the target-specific RNA, a self-splicing ribozyme and a DNA region involved in 3′ end formation and polyadenylation.

Construction of siRNA Libraries in Order to Silence Multitude of Genes

The present invention provides methods for constructing siRNA libraries comprising siRNAs that may suppress the expression of a subset of corresponding genes or a total repertoire of mRNAs in order to affect selectable cell phenotypes.

WO04101788 discloses methods for construction of random or semirandom siRNA libraries. U.S. Patent Application Publication no. 2005/0026172 discloses libraries for generating siRNA where the members of the library are optimized to inhibit the expression of genes that encode a predetermined family of proteins. Specific siRNA identified through this process may have direct therapeutic value.

Since the six bases of the poly(A) signal are common to most mRNAs, random siRNA libraries can be now constructed based on the AAUAAA site and the flanking variable sequences. This approach should diminish the size of siRNA random libraries and ensure effective silencing.

### Administration of Nucleic Acid Molecules to Host Cells

The short interference RNA can be chemically synthesized or expressed in a vector. A variety of different vectors are known in the art, including but not limited to a plasmid vector and a viral vector. Such vectors generally have convenient restriction sites located near the promoter sequence to provide for the insertion of nucleic acid sequences. Transcription cassettes can be prepared comprising a transcription initiation region, the target gene or fragment thereof, and a transcriptional termination region. The transcription cassettes can be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

Methods for the delivery of nucleic acid molecules are described in Akhtar et al., (Trends Cell Bio., 2, 139, 1992). WO 94/02595 describes general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example, through the use of conjugates and biodegradable polymers. More detailed descriptions of nucleic acid delivery and administration are provided for example in WO 93/23569, WO 99/05094, and WO 99/04819.

The nucleic acids can be introduced into tissues or host cells by any number of routes, including viral infection, microinjection, or fusion of vesicles. Jet injection may also be used for intramuscular administration, as described by Furth et al. (Anal. Biochem., 115 205:365-368, 1992). The nucleic acids can be coated onto gold microparticles, and delivered intradermally by a particle bombardment device, or “gene gun” as described in the literature (see, for example, Tang et al. Nature 356:152-154, 1992), where gold microprojectiles are coated with the DNA, then bombarded into skin cells.

The siRNA can be directly introduced into the cell (i.e., intracellularly); or introduced extracellularly into a cavity, interstitial space, into the circulation of an organism, introduced orally, etc. Methods for oral introduction include direct mixing of RNA with the food of the organism. Physical methods of introducing nucleic acids include injection directly into the cell or extracellular injection into the organism of an RNA solution. The agent can be introduced in an amount which allows delivery of at least one functional copy per cell. Higher doses (e.g., at least 5, 10, 100, 500 or 1000 or more copies per cell) of the agent may yield more effective inhibition; lower doses may also be useful for specific applications.

Other methods known in the art for introducing nucleic acids to cells can be used, such as lipid-mediated carrier transport, electroporation of cell membranes, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or otherwise increase inhibition of the target gene.

The expression of the RNA can be constitutive or regulatable. For example, the nucleic acid encoding the RNAi may be located on the vector where it is operatively linked to an appropriate expression control sequence e.g., the tetracyline repressor as described for example in International Patent Publication No. WO 04/065613.

To adapt RNAi for the study of gene function in animals, genetic engineering can be used to create mouse embryonic stem cells in which RNAi is targeted to a particular gene (Carmell et al., Nat Struct Bio1.10(2):91-92, 2003). This is based on a previous study in which silencing a gene of interest through RNAi was efficiently achieved by engineering a second gene that encoded short hairpin RNA molecules corresponding to the gene of interest (Carmell et al., 2003). The stem cells were injected into mouse embryos, and chimeric animals were born. Matings of these chimeric mice produced offspring that contained the genetically engineered RNAi-inducing gene in every cell of their bodies. It was observed from examination of the tissues from transgenic mice, that the expression of the gene of interest was significantly reduced throughout the organism (e.g. liver, heart, spleen). Such a reduction in gene expression is called a “gene knockdown” to distinguish it from traditional methods that involve “gene knockouts” or the complete deletion of a DNA segment from a chromosome. One advantage of this RNAi-based gene knockdown strategy, is that the strategy can be modified to silence the expression of genes in specific tissues, and it can be designed to be switched on and off at any time during the development or adulthood of the animal.

According to one embodiment of the present invention, the cells are transfected or otherwise genetically modified ex vivo. The cells are isolated from a mammal (preferably a human), nucleic acid introduced (i.e., transduced or transfected in vitro) with a vector for expressing an RNAi, and then administered to a mammalian recipient for delivery of the therapeutic agent in situ. The mammalian recipient may be a human and the cells to be modified are autologous cells, i.e., the cells are isolated from the mammalian recipient. According to another embodiment of the present invention, the cells are transfected or transduced or otherwise genetically modified in vivo. The cells from the mammalian recipient are transduced or transfected in vivo with a vector containing exogenous nucleic acid material for expressing an RNAi and the therapeutic agent is delivered in situ.

Recently, techniques have been developed to trigger siRNA into a specific target cell (e.g. embryogenic stem cell, hematopoietic stem cell, or neuronal cell) by introducing exogenously produced or intracellularly expressed siRNAs as described for example in WO 03/022052 and U.S. Patent Application 2005/0042646.

Depending on the nature of the RNAi agent, the active agent(s) can be administered to the host using any convenient means capable of resulting in the desired modulation of target gene expression. Thus, the agent can be incorporated into a variety of formulations for therapeutic administration. More particularly, the agents of the present invention can be formulated into pharmaceutical compositions by combination with appropriate, pharmaceutically acceptable carriers or diluents, and can be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intradermal, transdermal, intracheal, etc.

The RNAi may be introduced into plants by using any appropriate vector to transform the plant cell, applying methods such as direct gene transfer (e.g., by microinjection or electroporation), pollen-mediated transformation (as described, for example, in EP 270356, WO 85/01856 and U.S. Pat. No. 4,684,611), plant RNA virus-mediated transformation (as described, for example, in U.S. Pat. No. 4,407,956), liposome-mediated transformation (as described, for example, in U.S. Pat. No. 4,536,475), and the like.

Other methods, such as microprojectile bombardment are suitable as well. Cells of monocotyledonous plants, such as the major cereals, can also be transformed using wounded and/or enzyme-degraded compact embryogenic tissue capable of forming compact embryogenic callus, or wounded and/or degraded immature embryos as described in WO 92/09696. The resulting transformed plant cell can then be used to regenerate a transgenic plant in a conventional manner.

The obtained transgenic plant can be used in a conventional breeding scheme to produce more transgenic plants with the same characteristics or to introduce the expression cassette in other varieties of the same or related plant species, or in hybrid plants. Seeds obtained from the transgenic plants contain the expression cassette as a stable genomic insert.

Methods for Monitoring Efficacy of the siRNA

The consequences of inhibition can be confirmed by examination of the outward properties of the cell or organism or infectious agent (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed.

Having now generally described the invention, the same will be more readily understood through reference to the following examples, which are provided by way of illustration and are not intended to be limiting of the present invention.

### EXAMPLES

### Materials and Methods

(i) Cells:

Human cell lines HeLa and HEK293T (ATCC) were grown in DMEM supplemented with 10% fetal calf serum (FCS) and antibiotics. African green monkey cell lines CV1 (ATCC #CCL-70) and its derivate CMT4 (Gerared and Gluzman, 1985, Mol. Cell. Biol., 5, 3231-3240) were grown in DMEM supplemented with 10% FCS for CV1 and 5% for CMT4. Stable mouse Hepatoma C4 clones (b13Nbiil, ATCC, and CRL-2717) transfected with plasmids expressing a dominant positive, CREB300/310, and wild type CREB, respectively (Abramovitch et al., 2004, Cancer Res., 64, 1338-1346) were grown in DMEM supplemented with 10% fetal calf serum (FCS) and antibiotics.

(ii) Plasmids:

### Luciferase Expression Vectors:

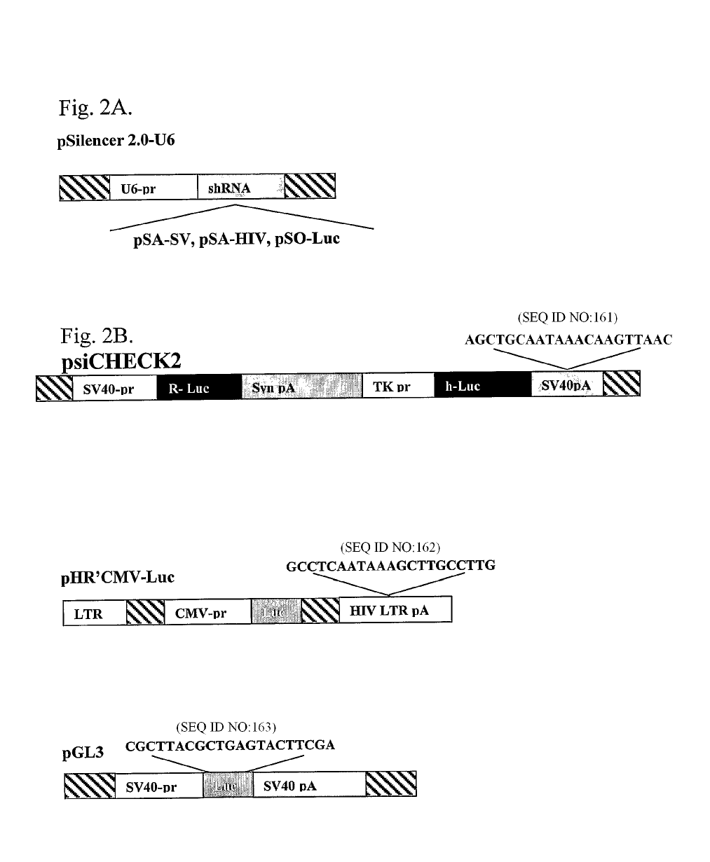

pHR-CMV-Luc (Naldini et al., 1996, Proc. Natl. Acad. Sci. USA, 93, 11382-11388), psiCHECK-2, pGL3 (FIG. 2A) and pRL-SV40 (Promega Corp.). pGLCRE-Hyg was generated by three fragment ligation: two fragments were generated by digestion of pGLCRE (Goren et al., 2001, J. Mol. Biol., 313, 695-709) with NcoI/SalI and Ncoi/XbaI and the last fragment was generated by digestion of pcDNA-hyg (Nitrogen) with XbaI/SalI. pBABE-Renilla was generated by inserting the Renilla Luc from pRL-SV40 digested with XbaI/HindIII into pBABE-Puro (Morgenstern et al., 1990, Nucleic Acids Res., 18, 3587-3596) digested with NheI/HindIII.

### Short Hairpin RNA (ShRNA) Expression Vectors:

The vector pSilencer 2.0-U6 (Ambion Corp.) served as the backbone for all constructs expressing the shRNA. All oligonucleotides for the expression of the shRNA were cloned between the BamHI and HindIII restriction sites on the vector. To construct the vector plasmid pSO-Luc which expresses a shRNA directed against a sequence in the open reading frame (ORF) of the luc gene, the following oligonucleotides were used:

Sense strand:

(SEQ ID NO: 155)

5′-GATCCCGCTTACGCTGAGTACTTCGATTCAAGAGATCGAAGTACTCA
GCGTAAGTTTTTTGGAAA,
and
Anti-sense strand:

(SEQ ID NO: 156)

5′-AGCTTTTCCAAAAAACTTACGCTGAGTACTTCGATCTCTTGAAGCGA
AGTACTCAGCGTAAGCGG.

For the construction of pSA-SV expressing an anti-SV40 poly (A) shRNA, the following oligonucleotides were cloned:

Sense strand:

(SEQ ID NO: 157)

5′GATCCCAGCTGCAATAAACAAGTTAACTTCAAGAGAGTTAACTTGTTT
ATTGCAGCTTTTTTTGGAAA;
and
Anti-sense strand:

(SEQ ID NO: 158)

5′-AGCTTTTCCAAAAAAAGCTGCAATAAACAAGTTAACTCTCTTGAAGT
TAACTTGTTTATTGCAGCTGG.

pSA-HIV expresses an anti-HIV poly(A) signal shRNA:

Sense strand:

(SEQ ID NO: 159)

5′-GATCCGCCTCAATAAAGCTTGCCTTGTTCAAGAGACAAGGCAAGCTT
TATTGAGGCTTTTTTGGAAA;
and
Anti-sense strand:

(SEQ ID NO: 160)

5′-AGCTTTTCCAAAAAAGCCTCAATAAAGCTTGCCCTTGTCTCTTGAAC
AAGGCAAGCTTTATTGAGGCG.

pGEM (Promega Corp.) was used to equilibrate DNA concentrations in all transfection experiments.

(iii) Luciferase Assay:

293T cells were transfected by calcium-mediated method with the following plasmid concentration: 1) 0.01 μg pHR-CMV-Luc/0.05 μg pRL-SV40 and different concentrations of pSA-HIV, 2) 0.2 μg psiCHECK-2 and different concentrations of pSA-SV.3. 0.01 μg pGL3/0.05 μg pRL-SV40 (Promega Corp.) and different concentrations of pSO-Luc. Hela cells were transfected by TransFast (Promega Corp.) with the following plasmid concentrations: 1) 0.1 g pHR-CMV-Luc/0.1 g phRL-SV40 and different concentrations of pSA-HIV, 2) 0.2 μg psiCHECK-2 and different concentrations of pSA-SV. 3) 0.5 μg pGL3/0.1 g phRL-SV40 and different concentrations of pSO-Luc. C4 cells were transfected by TransFast™ (Promega Corp.) with the following plasmid concentrations: 1 μg pGLCRE-Hyg, 0.5 μg pBABE-Renilla and different concentrations of pSA-SV. The pGEM plasmid (Promega Corp.) was used to equilibrate DNA concentrations. The cells were harvested 48 h after transfection into passive lysis buffer (Promega Corp.) and light emission was monitored by an automatic Anthos Lucyl photoluminometer. The Renilla Luc expressing vectors served as transfection controls. Firefly Luciferase activity was normalized to the activity of Renilla Luc expressed from either the cotransfected plasmid vector phRL-SV40 or psiCHECK-2 (see above). Results are presented as the percentage of Luciferase activity compared to activity in the absence of siRNA expressing vectors.

(iv) Northern Blot Analysis:

Total RNAs were isolated from Hela cells transfected by TransFast™ (Promega Corp., cat.#E2431) with pHR-CMV-Luc and the following siRNA expression plasmids:PSA-SVas control, pSo-Luc and pSA-HIV for Luciferase inhibition.

RNAs (8 μg) were subjected to electrophoresis on 1% agarose-formaldehyde gel and transferred to Nytran N (Schleicher & Schuell)—filters by diffusion blotting. The integrity of the RNA and the uniformity of RNA transfer to the membrane were determined by UV visualization of the ribosomal RNA bands in the gels and filters. The RNA was fixed by UV cross-linking. The RNA blots were hybridized to a random primed Luciferase cDNA derived from pGL3 (Promega Corp.) and β-actin cDNA.

(v) Western Blot Analysis:

Proteins were resolved on 4-12% gradient SDS-PAGE electrophoresis and then electrotransferred onto an Immobilon-P membrane (Millipore #IPVH00010) using Tris-Glycine buffer (20 mM Tris-base, 200 mM Glycine, 20% Methanol). The membrane was incubated in blocking buffer (1% casein, 0.4% Tween-20 in PBS), and reacted with rabbit polyclonal anti-VP1 (a gift from A. Oppenheim, the Hebrew University of Jerusalem) as first antibody, for 1 hr, followed by 3 washes with PBS for 5 minutes each. A second Anti-rabbit IgG antibody conjugate to HRP (Jackson IRL #111-035-003) was added in blocking buffer and incubated for 30 minutes at room temperature. After three washes with PBS the signal was developed by an ECL assay and membrane was exposed to film.

(vi) Assay for Viral Infectious Particles (IP):

The titer of SV40 IP was assayed on CMT4 cells (Dalyot-Herman et al., 1996, J. Mol. Biol., 259:69-80). Cells were infected with different virus dilutions. Viral DNA was allowed to replicate for 2-3 days. The cells were transferred onto nitrocellulose membrane, the DNA was denatured, fixed to the membranes and hybridized to a specific SV40 DNA probe.

(vii) Computational Analysis

poly(A) Specificity Analysis:

Human 3′-UTR transcripts, longer than 21 bases (total 19916) were retrieved from Ensebml, using the Ensmart tool.

For genes with more than a single transcript addition, all but one transcript were removed from the dataset. The data was further pruned to remove 3′-UTR not containing the canonic poly(A) signal AATAAA, leaving 13324 sequences. Sequences containing more than one occurrence of AATAAA were also removed, leaving 8477 sequences.

The poly(A) signal was extracted from those sequences, along with 10 bases upstream and 5 bases downstream, resulting in 8477 21-mers in which the signal AATAAA occupies positions 1′-16.

HIV Poly(A) Conservation Analysis:

HIV isolates were recovered from Entrez Nucleotide database, using the query string “hiv-1 complete genome”. 492 HIV sequences were retrieved.

All unique 21-bases-long sequences were extracted, and for each 21-mer, the number of genome containing it was counted.

### Example 1

### The Polyadenylation Signal of mRNAs as a Target for siRNA: Bioinformatic Analysis for Conservation and Uniqueness of the Poly(A) Region

Computational analysis of the human mRNA 3′UTR database was conducted in order to determine the uniqueness of sequences flanking the poly(A) signal (Table 2). Among the 8477 3′UTRs sequences containing one occurrence of AATAAA, 8477 mRNAs harbor a 21-mer unique sequence, including the AATAA, 10 bases upstream and 5 bases downstream. This signature can be used to uniquely specify each of these genes. This means that 97.4% of the genes in the dataset can be specifically recognized using their poly(A) region. The rest of the poly(A) regions, are shared among several genome locations, of which at least 25% are annotated to be producing the same protein. Many of the others belong to different genes that produce different proteins, but belong to the same protein family, e.g. the two genes: WILLIAMS BEUREN SYNDROME CHROMOSOME REGION 20C ISOFORM 1 and WILLIAMS-BEUREN SYNDROME CRITICAL REGION PROTEIN 20 COPY B. These results indicate that the poly(A) signal and its flanking sequences may serve as a general and yet specific target for siRNA.

TABLE 2

Poly(A) signal region^{a}
In human 3′ UTR dataset

| No. | % | Occurrence |
|---|---|---|
| 8261 | 97.45 | 1 |
| 75 | 0.88 | 2 |
| 8 | 0.09 | 3 |
| 5 | 0.06 | 4 |
| 1 | 0.01 | 5 |
| 0 | 0 | 6 |
| 1 | 0.01 | 7 |
| 0 | 0 | 8 |
| 0 | 0 | 9 |
| 1 | 0.01 | 10 |

^{a}poly(A) signal region refer to the canonical poly(A) signal AAUAAA along with 10 bases upstream and 5 bases downstream.

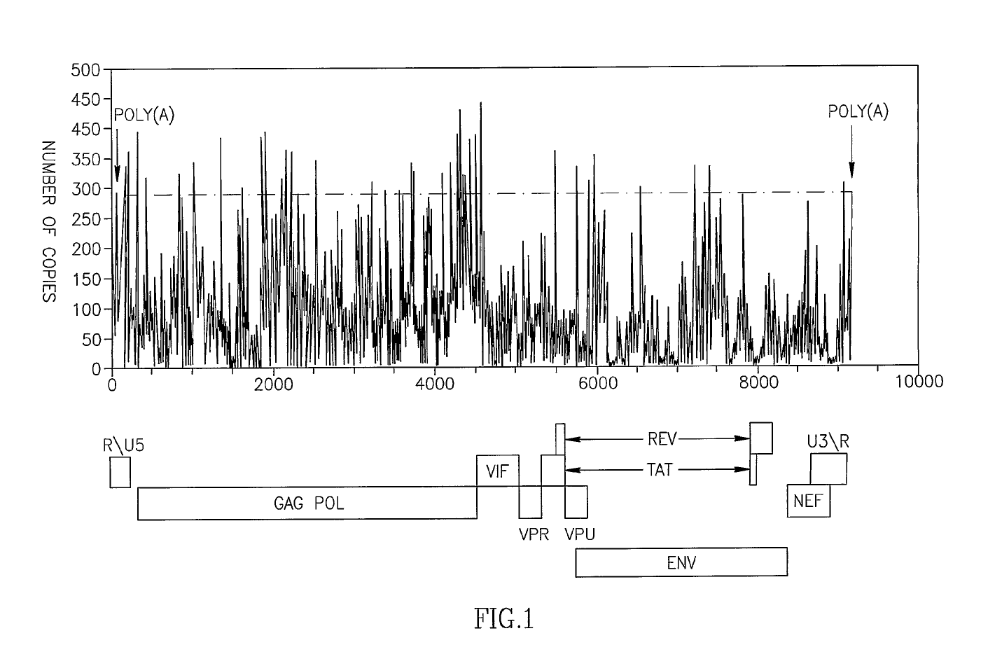

It is well known that the HIV 1 genome undergoes frequent mutagenesis and several of its regions are mutation tolerable. Indeed, the results of the computational analysis for conservation of 21 bases along the genome of HIV 1 presented in FIG. 1, demonstrate that the 21 bases including the AAUAAA poly(A) signal is highly conserved in 285 out of 492 tested genomes (58%), falling into the highest 8% of conserved HIV 1 sequences.

### Example 2

### The Polyadenylation Signal of mRNAs as a Target for siRNA

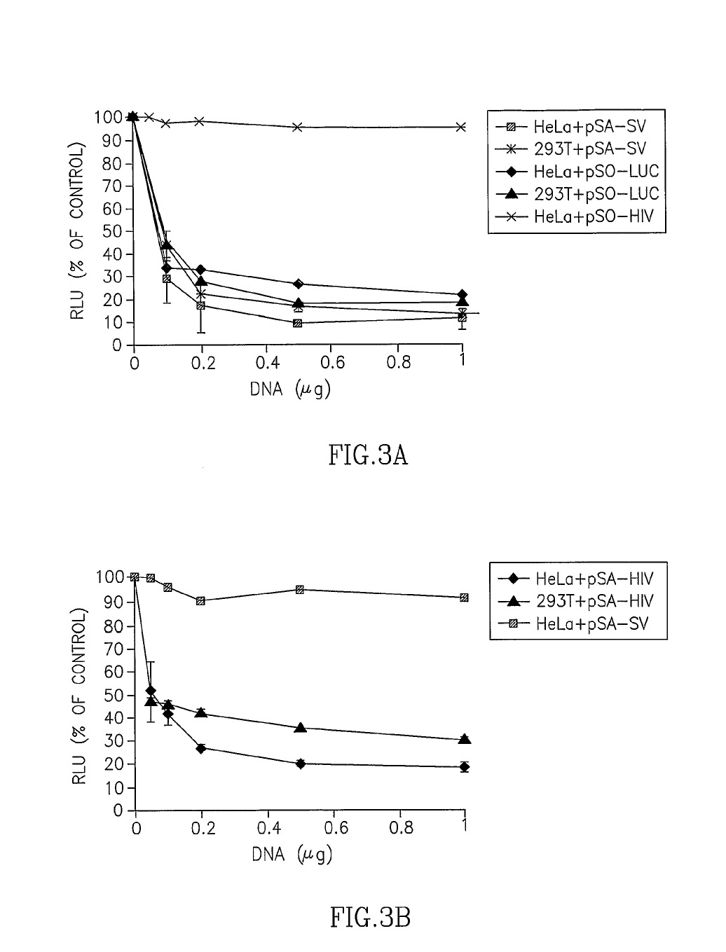

To test experimentally if the poly(A) region is indeed an efficient target for siRNA silencing, vectors that express a 21 bases long shRNA, homologous to the poly(A) region, that include, the AATAAA sequence, five bases upstream and ten bases downstream, were constructed (FIG. 2A). These shRNA expression plasmid vectors were co-transfected into HeLa and 293T cells with vectors in which the RNA of the luc gene is processed at the 3′ end at either a SV40 or a HIV-1 poly(A) signal (FIG. 2B). As a control, cells were co-transfected with a vector in which the Renilla Luciferase (R-luc) RNA is processed at a synthetic poly(A), nonhomologous to either one of the two siRNAs (FIG. 2B). In experiments that targeted the shRNA to the SV40 poly(A) region (anti-SV40 poly(A) signal shRNA (SEQ ID NO: 161)), the vector pSA-SV, was cotransfected together with the psiCHEK2, in which the luc RNA is processed at the SV40 poly(A) signal, and the control R-luc gene is processed at a synthetic poly(A) site (FIG. 2B). Luciferase activity was monitored in cell lysates 48 hrs following the transfection. Levels of Luc activity were normalized to the activity of Renilla Luciferase expressed in the same cells. Specific silencing, in a dose response manner, was observed in both HeLa and 293 cell lines, reaching a maximal inhibition of 88% (FIG. 3A). Similar results were observed when targeting the HIV poly(A) signal using an anti-HIV poly(A) signal shRNA (SEQ ID NO: 162). In the latter experiments the cells (HeLa and 293T) were cotransfected with pSA-HIV together with pHR′CMV-Luc, in which luc RNA is processed at the HIV poly(A) signal, and pRL-SV40 in which the control R-luc gene is processed at a SV40 poly(A) site (FIG. 3B).

Efficiency of silencing by siRNA directed against the poly(A) signal was compared to the knock-down by siRNA directed to an internal coding sequence of the luc mRNA (pSO-Luc, FIG. 2A), previously shown to be a very sensitive site for siRNA silencing. In this experiment the luc mRNA expressed from plasmid vector pGL3 served as a target for the siRNA and the Renilla Luciferase mRNA expressed from pRL-SV40 served as a negative control (an anti-Luc ORF.shRNA sequence (SEQ ID NO: 163) The results presented in FIG. 3A clearly indicate that targeting the poly(A) site abrogates gene expression as effectively as targeting a known sensitive internal coding sequence (88% inhibition).

To further validate the specificity of the poly(A) region for knockdown, two reciprocal experiments were carried out. In one experiment cells were co-transfected with pHR′CMV-Luc, in which the luc RNA is processed at the HIV poly(A) site and with pSA-SV, expressing siRNA directed against the SV40 poly(A) region. In a reciprocal experiment the cells were cotransfected with psiCHECK2 (luc RNA processed at the SV40 poly(A)) and pSA-HIV (siRNA directed to the HIV poly(A) region). In both experiments there was no inhibition of Luciferase activity controlled by the heterologous poly(A) (FIGS. 3A and 3B). It is interesting to note that although luc gene expression was higher by two orders of magnitude, in the 293 cells, compared to HeLa cells, the inhibition of Luciferase expression was similar in both cell lines. This observation further indicates the efficiency of targeting the poly(A) site of mRNAs by specific siRNAs.

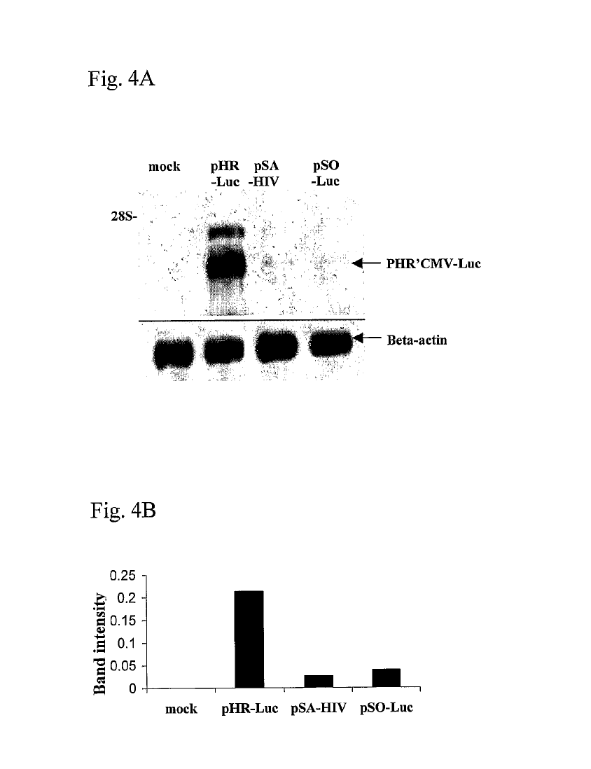

The knockdown of Luc activity by siRNA was verified by direct quantification of the mRNAs levels in the transfected cells (FIG. 4). HeLa cells co-transfected with pHR′CMV-Luc and pSA-HIV, producing siRNA that targets the HIV poly(A), or with pSO-Luc expressing siRNA targeting the luc ORF, were subjected to northern blot analysis. Indeed, a reduction of ten fold in luc mRNA levels (measured, by RNA band intensity and normalized to β-actin mRNA level in the same RNA sample) was observed relative to the control cells transfected with a non-relevant shRNA expressing-vector (pSA-SV, FIG. 4).

In conclusion, siRNA directed to the poly(A) signal region of SV40 or HIV-1 specifically and efficiently reduced, gene expression, mediated by mRNA degradation, in two different cell lines and two different viral targets.

### Example 3

### siRNA Directed Inhibition of SV40 Late Proteins and Viral Replication

The SV40 circular dsDNA chromosome is transcribed from two promoters controlling the expression of the early and late viral functions. The 3′ end processing of each of these two transcripts is controlled by a different poly(A) signal. To determine whether vectors expressing siRNA directed against the SV40 poly(A) region can inhibit viral propagation, cells were co-transfected with SV40 complete genome DNA and pSA-SV (targeting the SV40 late poly(A) region). The cell cultures were lysed seventy-two hours following transfection and proteins were resolved by PAGE and subjected to Western blot analysis. Antibodies specific for the SV40 VP1 capsid protein were used for the detection of VP1. In cells co-transfected with pSA-SV, the VP1 protein level was 16 fold lower then in the control cells, co-transfected with a non-relevant siRNA construct (FIG. 5A).

Next, it was analyzed whether siRNA mediated inhibition of the SV40 late gene also affect SV40 virus replication. Cells, CMT4, were co-transfected with SV40 DNA and pSA-SV or with SV40 DNA and a non-relevant siRNA vector (pSO-Luc). At 3, 4 and 5 days post transfection, progeny virus was harvested, diluted and quantified by infection of CMT4 cells. Two days after infection the cell were lysed and cell extracts were blotted onto a nitrocellulose membrane and subjected to plaque hybridization, using a SV40 specific DNA probe. Virus titers dropped dramatically, by 87%, in cells co-transfected with the pSA-SV, relative to the control transfection (FIG. 5B). These results further demonstrate that siRNA directed against the poly(A) of viruses efficiently inhibit viral propagation.

### Example 4

### Specific Inhibition of a Stable Transgene Expression

In the experiments described above it was demonstrated that the poly(A) region can serve as a sensitive target for siRNA silencing in transient co-transfection experiments. To further elucidate the efficiency of siRNA targeted to the poly(A) region of genes integrated in the chromosome we utilized a hepatoma cell line C4. The knock down of CREB (human Cyclic AMP Responsive Element Binding protein,) mRNA was tested on two cells lines: one expressing the wild type CREB, and the second expressing a positive dominant CREB mutant CREB300/310 (Goren et al., 2001, J. Mol. Biol., 313, 695-709). In both cell lines the 3′ end of the recombinant CREB mRNA is generated at the SV40 poly (A) site. To determine the level of CREB knock-down, the cells were cotransfected with three plasmids: The siRNA expressing vector pSA-SV, the luciferase reporter plasmid pGLCRE-Hyg, in which initiation of transcription of the luc gene is controlled by the CRE consensus sequence, and with a control non-CREB dependent Renilla luc expressing plasmid (pBABE-Renilla, see materials and methods).

The level of Luc activity measured in the C4 control cells, not expressing any recombinant CREB, served as a base line, indicating the basal activation of the CRE promoter by the endogenous m-CREB of the cells. The shRNA directed against the SV40 poly(A) signal is not expected to knock-down the mRNA of the endogenous CREB gene since it has its own unique poly(A) region. Indeed, the CRE mediated basal Luc activity, measured in these cells, is not affected by siRNA expressed by the pSA-SV vector (FIG. 6). On the other hand, luc expression in cells which stably express the recombinant h-CREB variants, controlled by the SV40 poly(A) signal, showed a marked decrease of the CRE mediated luciferase activity. The low level of luc expressed in the presence of SiRNA in these cells was similar to the background level mediated by the endogenous m-CREB (FIG. 6). The results of this experiment demonstrate that knock-down of chromosomal genes via the poly(A) signal region is possible and efficient. Moreover, the results indicate that it is possible to specifically knockdown an endogenous gene without effecting an exogenous copy of the same gene and vise versa.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A method for the production of a small interference RNA (siRNA) molecule for silencing the expression of a specific gene having AAUAAA as a polyadenylation signal site sequence, the method comprising the steps of:
a) identifying an oligonucleotide sequence of the specific gene, wherein said oligonucleotide sequence is about 15 to about 40 nucleotides in length and comprises
(i) said polyadenylation signal site sequence and
(ii) unique non-coding sequences flanking said polyadenylation signal site; and
b) synthesizing oligonucleotide molecules having said oligonucleotide sequence (a), thereby obtaining an siRNA molecule for silencing said specific gene.

2. The method of claim 1, wherein said unique non-coding sequences are 9-34 nucleotides in length.

3. The method of claim 1, wherein the orientation of said unique non-coding sequences with respect to said polyadenylation signal site sequence is selected from the group consisting of adjacent 5′ sequence, adjacent 3′ sequence, and combinations of adjacent 5′ and 3′ sequences.

4. The method of claim 1, wherein said unique non-coding sequences provide specificity of said siRNA molecule to said specific gene.

5. The method of claim 1, wherein said siRNA molecule comprises from about 15 to about 40 nucleotides.

6. The method of claim 1, wherein said siRNA molecule comprises from about 18 to about 25 nucleotides.

7. The method of claim 1, wherein said siRNA molecule is designed by a bio-informatic program to predict the optimal length of said unique non-coding sequences to be used on either end of the consensus sequence of said polyadenylation signal site sequence.

8. The method of claim 1, wherein said siRNA molecule is capable of inhibiting the expression of said specific gene in a cell.

9. The method of claim 8, wherein said specific gene is an endogenous cellular gene.

10. The method of claim 8, wherein said specific gene is an exogenous gene, not present in the normal cellular genome.

11. The method of claim 8, wherein said specific gene is a viral gene.

12. The method of claim 8, wherein said specific gene is of mammalian origin, avian origin or plant origin.

13. The method of claim 8, wherein said specific gene is of human origin.

14. The method of claim 8, wherein said specific gene is expressed in a tumor cell.

15. The method of claim 8, wherein the expression of said specific gene is inhibited by at least 50% by said siRNA.

16. The method of claim 8, wherein said siRNA molecule inhibits virus propagation.

17. The method of claim 8, wherein said siRNA molecule inhibits cell proliferation.

18. The method of claim 8, wherein the sequence of said siRNA molecule includes at least one mismatch pair of nucleotides.

19. The method of claim 18, wherein the sequence of said siRNA molecule includes no more than two mismatch pairs of nucleotides.

20. A pharmaceutical composition comprising as an active ingredient a short interference RNA (siRNA) molecule according to method of claim 1, and a pharmaceutically acceptable carrier.
